(19) United States
(12) Patent Application Publication
(10) Pub. No.: US 2013/0197023 A1
(43) Pub. Date: Aug. 1, 2013

(54) NEUROPROTECTION BY PHARMACOLOGICAL CHAPERONING OF NICOTINIC ACETYLCHOLINE RECEPTORS

(71) Applicants: Henry A. Lester, So. Pasadena, CA (US); Rahul Srinivasan, Pasadena, CA (US); Christopher I. Richards, Lexington, KY (US); Brandon J. Henderson, Pasadena, CA (US)

(72) Inventors: Henry A. Lester, So. Pasadena, CA (US); Rahul Srinivasan, Pasadena, CA (US); Christopher I. Richards, Lexington, KY (US); Brandon J. Henderson, Pasadena, CA (US)

(73) Assignee: California Institute of Technology, Pasadena, CA (US)

(21) Appl. No.: 13/752,228

(22) Filed: Jan. 28, 2013

Related U.S. Application Data
(60) Provisional application No. 61/591,737, filed on Jan. 27, 2012.

(51) Int. Cl. A61K 31/465; A61K 31/4745; A61K 31/439

(52) U.S. Cl. CPC A61K 31/465; A61K 31/439; A61K 31/4745
USPC 514/288; 514/343; 514/292; 435/375

(57) ABSTRACT

A method of ameliorating an endoplasmic reticulum (ER) stress response and/or unfolded protein response (UPR) in cells expressing nicotinic acetylcholine receptors (nAChRs) comprises contacting a cell expressing nAChRs with an effective amount of a ligand for nAChRs. The contacting results in attenuating endogenously expressed ATF6 translocation, expression of XBP1, phosphorylation of eukaryotic initiation factor 2α (peIF2α), increased numbers of ER exit sites, increased trafficking of known associated proteins as well as other proteins such as growth factors and their receptors, changes in abundance of selected mRNA species, and phosphorylation, abundance, or subcellular compartmentalization of other proteins involved with ER stress and/or the UPR, and inhibiting upregulation of CCAAT/Enhancer-Binding Protein Homologous Protein (CHOP) levels in the cells. The method can be used to screen for neuropharmacotherapeutic agents and to treat or prevent neurodegenerative disease, such as amyotrophic lateral sclerosis (ALS), Parkinson's disease, Alzheimer's disease, or cognitive deficiency.

This application claims the benefit of U.S. provisional patent application No. 61/591,737, filed Jan. 27, 2012, the entire contents of which are incorporated herein by reference.

### STATEMENT REGARDING FEDERALLY SPONSORED RESEARCH

This invention was made with government support under AG033954, DA011729, MH086386, NS011756 awarded by the National Institutes of Health. The government has certain rights in the invention.

### TECHNICAL FIELD OF THE INVENTION

The present invention relates to agents and methods for protection of neural cells from adverse events such as the endoplasmic reticulum (ER) stress response and the unfolded protein response (UPR). More specifically, the invention relates to ligands for nicotinic acetylcholine receptors (nAChRs) and their use in protecting neural cells from apoptosis, thereby providing a means to treat, prevent or delay the progression of neurodegenerative diseases.

### BACKGROUND OF THE INVENTION

Nicotine appears to cause at least part of the well-documented inverse correlation between a person's history of smoking and his/her susceptibility to developing Parkinson's disease (PD) (Hernan et al., 2002; Lester et al., 2009; Quik et al., 2011). The use of smoked or smoke-cured tobacco has no medical justification. Therefore it is essential to understand the mechanistic basis for the apparent neuroprotective action of nicotine.

Nicotine binds to and activates neuronal nicotinic acetylcholine receptors (nAChRs), a family of ligand-gated ion channels comprising homo- and heteropentameric combinations of α (α2 to α10) and β (β2 to β4) subunits (Gotti et al., 2007). The substantia nigra pars compacta (SNc) dopaminergic neurons (DA) that degenerate in PD robustly express several nAChR receptor subtypes; some of these subtypes are substantially retained within the endoplasmic reticulum (ER) (Azam et al., 2002; Commons, 2008; Hill et al., 1993), rather than becoming efficiently trafficked to the plasma membrane. Long-term activation of the unfolded protein response (UPR) can lead to apoptosis (Kim et al., 2006; Li et al., 2006). Indeed, postmortem studies have shown that DA neurons in PD patients display an increase in UPR markers (Hoozemans et al., 2007).

The best-known effect of chronic exposure to nicotine is to upregulate nAChRs in vivo and in vitro via cell-delimited, post-translational mechanisms (Buisson and Bertrand, 2002; Gopalakrishnan et al., 1997; Lester et al., 2009; Nashmi et al., 2007; Peng et al., 1994; Sallette et al., 2005; Schwartz and Kellar, 1983; Srinivasan et al., 2011). Emerging data suggest that selective pharmacological chaperoning of acetylcholine receptor number and stoichiometry (SePhaChARNS) by nicotine underlies nicotine-induced nAChR upregulation (Kuryatov et al., 2005; Lester et al., 2009; Miwa et al., 2011; Sallette et al., 2005; Srinivasan et al., 2011).

There remains a need for methods and agents that can attenuate the ER stress response and UPR, and that can provide neuroprotective effects to treat and prevent neurodegenerative disease.

### SUMMARY OF THE INVENTION

The invention provides a method of ameliorating an endoplasmic reticulum (ER) stress response and/or unfolded protein response (UPR) in cells expressing nicotinic acetylcholine receptors (nAChRs). Typically, the contacting comprises intracellular binding of the ligand to nAChRs in endoplasmic reticulum or cis-Golgi apparatus. The method comprises contacting a cell expressing nAChRs with an effective amount of a ligand for nAChRs. In a typical embodiment, the contacting results in attenuating endogenously expressed ATF6 translocation, expression of XBP1, phosphorylation of eukaryotic initiation factor 2α (peIF2α), and/or increased numbers of ER exit sites. These are representative intracellular events triggered in response to ER stress and/or an accumulation of unfolded or misfolded proteins in the cytoplasm. Other examples of cellular events triggered by the ER stress response and/or UPR include, but are not limited to, increased trafficking of known associated proteins as well as other proteins such as growth factors and their receptors, changes in abundance of selected mRNA species, and phosphorylation, abundance, or subcellular compartmentalization of other proteins involved with ER stress and/or the UPR. In some embodiments, amelioration of the ER stress response comprises inhibiting upregulation of CCAAT/Enhancer-Binding Protein Homologous Protein (CHOP) levels in the cells.

In one embodiment, the cells are central nervous system (CNS) neurons. Representative examples of such neurons include, but are not limited to, thalamic, cortical, habenular and substantia nigra (SN) neurons. Also included are upper motor neurons and lower motor neurons. Typically, the ligand is an α4β2 agonist, and may be a full or partial agonist. In some embodiments, the ligand is an allosteric modulator. Examples of agonists include: cytisine, varenicline, galanthamine, CP-601927, ABT-089, A-85380, (±)-epibatidine, (−)-nicotine, lobeline, sazetidine-A, and derivatives thereof.

In one embodiment, the cells are in a patient having, or at risk of developing, a neurodegenerative disorder. Alternatively, the cells may be ex vivo or in vitro. The invention additionally provides a method of treating, preventing, or delaying the onset of, a neurodegenerative disorder in a subject. The method comprises administering to the subject an α4β2 agonist. Examples of neurodegenerative disorders include, but are not limited to, Alzheimer's disease, Huntington's disease, spinal muscular atrophy, Parkinson's disease, Alzheimer's disease, amyotrophic lateral sclerosis (ALS), progressive muscular atrophy (PMA), progressive bulbar palsy (PBP), primary lateral sclerosis (PLS), Creutzfeldt-Jakob disease, primary progressive aphasia, progressive supranuclear palsy, retinitis pigmentosa, macular degeneration, and cognitive deficiency.

The administering of the ligand (or the contacting of ligand with cells in vivo) comprises transdermal administration, oral administration, intrathecal administration, intramuscular administration, intraperitoneal administration, intranasal administration, intravenous administration or subcutaneous administration. In a typical embodiment, the effective amount of ligand is less than the amount required to activate nAChRs. The contacting can occur for 48 hours, for at least two weeks, or in some embodiments, for at least two months.

The invention also provides a method of screening for neural pharmacotherapeutic agents. In one embodiment, the method comprises contacting a candidate agent with a cell modified to express an α4β2 nAChR; and measuring an indicator of ER stress. The indicator can be selected from expression and/or translocation in the cell of AFT6, phosphorylation of eIF2α, and expression of XBP1, reduced upregulation of CHOP, and measuring ER exit sites, for example. The method of screening can further comprise introduction of a UPR activator, such as tunicamycin. This addition to the method allows for identifying agents that attenuate the stress response to a UPR activator. In one embodiment, the cell is pre-treated with the candidate agent prior to introduction of the UPR activator, to allow time for a potentially cumulative protective effect of the agent.

### BRIEF DESCRIPTION OF THE FIGURES

FIG. 1. Schematic illustration contrasting “outside-in” and “Inside-out” pathways at nAChRs. In the latter, nicotine enters the neuron, permeates into the ER and serves as a chaperone that favors assembly and stabilization of α4β2 nAChRs (insert at bottom). This decreases interactions with BiP, modulating PERK-ATF4 and IRE1-XBP1 (also shown in the insert at bottom). The insert at top shows the ATF6 cleavage/translocation pathway, which is also reduced by nicotine. The IRE1 branch of the UPR is shown as a dashed line. The M3-M4 loop of some nAChR subunits mediates ER retention and export via interactions with COPI and COPII proteins (middle insert).

FIGS. 2A-2C. Demonstration that each of the four manipulations (full agonist, partial agonist, antagonist and mutant subunit) decreases ATF6 translocation in Neuro-2a cells. FIG. 2A shows representative confocal images of Neuro-2a cells expressing α4-mcherryβ2-wt nAChRs+ATF6-eGFP or only ATF6-eGFP. Scale bars, 10 μm. FIG. 2B is a bar graph showing the ratio of fluorescence intensity for ATF6-eGFP (nucleus/whole cell section). Treatment conditions and transfected subunits are indicated on the x-axis. DM is the double mutant β2_{enhanced-ER-export} subunit. FIG. 2C is a bar graph showing the ratio of fluorescence intensity for ATF6-eGFP (nucleus/whole cell section) in the absence of co-expressed nAChRs. Drug treatments are indicated on the x-axis. p values are based on a 2-tailed t-test (** p<0.01, *** p<0.001). Data were obtained from 30 to 40 cells imaged for each condition.

FIGS. 3A-3D. Nicotine inhibits ATF6 translocation to the nucleus and eIF2α phosphorylation in mouse cortical neurons. FIG. 3A presents representative confocal images of mouse cortical neurons expressing α4-mcherryβ2-wt and immunostained for endogenous ATF6, peIF2α or total eIF2α. FIG. 3B is a bar graph showing the fluorescence intensity ratios of endogenously expressed ATF6 with and without 24 h, 0.1 μM nicotine (Nic) treatment. FIG. 3C is a bar graph showing the nuclear fluorescence intensity for peIF2α with and without 24 h, 0.1 μM nicotine (Nic) treatment. FIG. 3D is a bar graph showing the fluorescence intensity of total eIF2α. Bars show nicotine treatment conditions in parenthesis. The subcellular compartments imaged are indicated on the X-axis. p values are based on a 2-tailed t-test (* p<0.05, ** p<0.01). Data were obtained from 20 to 30 cells imaged for each condition.

FIGS. 4A-4C. Exposure to all three nicotinic ligands increases the formation of ER exit sites (ERES). FIG. 4A presents representative confocal images of a Neuro-2a cell expressing α4-mcherryβ2-wt nAChRs and Sec24D-eGFP (ERES marker). Scale bars, 10 μm. FIG. 4B presents an image of the same cell with ERES demarcated for quantification. FIG. 4C is a bar graph showing the quantification of average ERES fluorescence intensity. Drug treatment conditions are indicated on the x-axis. Error bars are ±SEM. p values are based on a 2-tailed t-test (* p<0.05, ** p<0.01). Data were obtained from 30 to 40 cells imaged for each condition.

FIGS. 5A-5E. The three nicotinic ligands have diverse effects on ER and trans-Golgi architecture. FIG. 5A presents representative TIRFM images of a cell co-expressing α4-eGFPβ2-wt nAChRs and pDSred2-ER marker. Merge shows nearly complete co-localization of fluorescence from nAChRs and DSred2-ER. Scale bars, 10 μm. FIG. 5B is a bar graph showing quantification of average ER area obtained using DSred2-ER fluorescence. Drug treatments are indicated on the x-axis. FIG. 5C presents representative TIRFM images of a cell expressing α4-eGFPβ2-wt nAChRs and GaIT-mcherry. Merge shows co-localization of GaIT-mcherry with nAChRs. Scale bars, 10 μm. FIG. 5D is a bar graph showing quantification of the number of TGN bodies visualized using GaIT-mcherry under each treatment condition, indicated on the x-axis. FIG. 5E is a bar graph showing quantification of the average TGN body fluorescence intensity visualized using GaIT-mcherry under each treatment condition, indicated on the x-axis. Error bars are ±SEM. p values are based on a 2-tailed t-test (* p<0.05, ** p<0.01). Data were obtained from 30 to 40 cells imaged for each condition.

FIGS. 6A-6B. The three nicotinic ligands have diverse effects on α4β2 nAChR stoichiometry. NFRET measurements using α4-mcherry and β2-eGFP subunits transfected into Neuro-2a cells. Columns in FIG. 6A indicate the regions of interest (whole cell or TG+TGN) and rows indicate drug treatment conditions. For each graph, the blue curve is the overall fit and dashed line Gaussians are individual fits. The fractional area of low-FRET pixels (FA), corresponding to the (α4)2(β2)3 stoichiometry is shown for each graph. FIG. 6B presents representative NFRET images of Neuro-2a cells. Drug treatments are indicated for each cell. Calibration bars are from zero to 20%. Scale bars, 5 μm.

FIGS. 7A-7D. The three nicotinic ligands have diverse effects on plasma membrane-localized α4β2 nAChRs. FIG. 7A presents representative traces showing whole-cell currents induced by puffs of 0.1, 500 μM nicotine and 0.1 μM cytisine, 500 μM nicotine. FIG. 7B presents representative TIRFM images of cells expressing α4-eGFPβ2-wt and treated with indicated drugs for 48 h. Scale bars, 10 μm, FIG. 7C is a bar graph showing normalized PM fluorescence intensity from TIRF images. Drug treatment conditions are shown on the x-axis. Error bars are 99% confidence interval. FIG. 7D is a bar graph of normalized footprint/ER ratios from TIRF images. Drug treatment conditions are shown on the x-axis. Error bars are ±RSE. Data were obtained from 30 to 40 cells imaged for each condition.

FIGS. 8A-8C. Demonstration of the presence of endogenous neuronal nicotinic acetylcholine receptors (nAChRs) in cultured mouse dopaminergic neurons. FIG. 8A shows an image of a 3 week old mouse ventral midbrain culture obtained from 14 day old mouse embryos. The white arrow points to a TH positive dopaminergic neuron and the black arrow points to a TH negative GABAergic neuron. FIG. 8B shows representative images of cultured dopaminergic neurons obtained from a transgenic mouse with a mcherry fluorescent protein-tagged α4 nAChR subunit. TH positive neurons show that these neurons express α4* nAChRs. TH positive neurons from parallel cultures of wildtype mouse embryos do not show mcherry fluorescence. FIG. 8C shows representative images of a midbrain dopaminergic neuron obtained from a transgenic mouse with an eGFP fluorescent protein-tagged α6 nAChR subunit, immunostained for eGFP, and a wildtype control. α6* nAChRs are present intracellularly, within the cell soma and the neurites. Control cultures obtained from wildtype mice do not show eGFP fluorescence. The haze in wildtype neurons is non-specific staining by the secondary antibody.

FIGS. 9A-9B. Demonstration that chronic exposure to nicotine prevents the upregulation of ER stress markers, ATF6, XBP1 and CHOP in cultured dopaminergic neurons treated with Tunicamycin, a known inducer of the ER stress response. FIG. 9A presents representative confocal images of dopaminergic neurons showing TH staining (primarily visible in the cytoplasm) and the indicated ER stress marker (primarily visible in the nucleus). The white line shows manually demarcated nuclei for each neuron. FIG. 9B presents graphs of average nuclear intensity from neurons in DMSO, tunicamycin (Tu) or nicotine (Nic) and tunicamycin (Tu) treated culture dishes. The ER stress marker that is quantified is indicated at the top of each graph and the concentration and time points for nicotine and tunicamycin treatments are indicated at the bottom of each graph. Numbers of cells imaged are indicated in parentheses for each column and p values are based on a 2-tailed t-test; error bars are ±S.E.M.

### DETAILED DESCRIPTION OF THE INVENTION

The present invention is based on the discovery and identification of molecules and methods for protection of neural cells from adverse events such as the endoplasmic reticulum (ER) stress response and the unfolded protein response (UPR). More specifically, the invention relates to ligands for nicotinic acetylcholine receptors (nAChRs) that bind intracellular receptors, such as to nAChRs in endoplasmic reticulum or cis-Golgi apparatus. Intracellular binding of these ligands can be employed in methods of protecting neural cells from apoptosis, thereby providing a means to treat, prevent or delay the progression of neurodegenerative diseases. The invention is further based on an hypothesis that changes in the accumulation of nAChRs within the ER of DA neurons can influence a possible UPR/ER stress response.

The molecules and methods disclosed herein overcome problems and limitations associated with other methods previously attempted that focus on activation of nAChRs without appreciating the effect of chronic, low-level exposure to ligand on the ER stress response. For example, agents that serve as pharmacological chaperones can protect cells from an apoptotic stress response. Moreover, in contrast with the general consensus in the field of ER stress, the data presented herein show that the XBP1 pathway may be pro-apoptotic in dopaminergic neurons. Accordingly, means of attenuating the XBP1 pathway under ER stress conditions may be protective against neurodegeneration.

### DEFINITIONS

All scientific and technical terms used in this application have meanings commonly used in the art unless otherwise specified. As used in this application, the following words or phrases have the meanings specified.

As used herein, an “nAChR” means capable of binding ligand with a binding constant typically less than 10 μM. Binding constants (Ki values) can be determined by using a competitive binding assay with, for example, ³H-epibatidine. It is understood that binding constants vary with the specific method, salt concentration, incubation time, and other variables. The affinity may also vary with the subcellular localization of the nAChR.

As used herein, “α4β2*” refers to heteropentameric nicotinic acetylcholine receptors reported to be involved in cognition and neuroprotection both in animals and humans. These receptors are widely expressed in human brain (reviewed in Bencheri, M. and J. D. Schmitt, Current Drug Targets Volume 1, number 4, August 2002; pp 349 357—note that entire issue is dedicated to nicotinic receptor distribution and effects). Accordingly, the terms “nAChR” and “nicotinic receptor,” as used herein, encompass such receptors including these subunits. The * in the expression “α4β2*” includes the possibility that other subunits such as α5, β3, and or α6 are present. Specifically, “α6β2*” receptors are among those contemplated. These latter receptors are expressed in dopaminergic neurons.

An “agonist” is a substance that stimulates its binding partner, typically a receptor. Stimulation is defined in the context of the particular assay, or may be apparent in the literature from a discussion herein that makes a comparison to a factor or substance that is accepted as an “agonist” or “partial agonist” of the particular binding partner by those of skill in the art. Stimulation may be defined with respect to an increase in a particular effect or function that is induced by interaction of the agonist or partial agonist with a binding partner and can include allosteric effects.

An “antagonist” is a substance that inhibits its binding partner, typically a receptor. Inhibition is defined in the context of the particular assay, or may be apparent in the literature from a discussion herein that makes a comparison to a factor or substance that is accepted as an “antagonist” of the particular binding partner by those of skill in the art. Inhibition may be defined with respect to an decrease in a particular effect or function that is induced by interaction of the agonist with a binding partner, and can include allosteric effects.

As used herein, a “derivative” of an agonist is a chemical substance produced from another agonist either directly or by modification or partial substitution, and having the same or higher affinity for the same receptor. Examples of cytisine derivatives and means for identifying those of particular utility are described in Nicolotti, O. et al., Farmaco, 2002 June; 57(6):469-78.

As used herein, an “endoplasmic reticulum stress response” or an “unfolded protein response” refers to intracellular events triggered in response to stress and/or an accumulation of unfolded or misfolded proteins in the cytoplasm. Examples of such events include activation of one or more of three pathways: (1) the ATF6 pathway, which influences genes for XBP1, ER quality control genes, and lipid synthesis; (2) The PERK pathway, which modifies transcription of genes encoding amino acid transporters and redox enzymes; and (3) The IRE1 pathway, which modifies genes for ER chaperones, ERAD, lipid synthesis, and ERO1. These pathways are modulated by ligands for the nAChRs.

As used herein, a “neurodegenerative disorder” means a disorder characterized by apoptotic neuronal death and loss of neurological function. Examples of neurodegenerative disorders include, but are not limited to, Alzheimer's disease, Huntington's disease, spinal muscular atrophy, Parkinson's disease, Alzheimer's disease, amyotrophic lateral sclerosis (ALS), progressive muscular atrophy (PMA), progressive bulbar palsy (PBP), primary lateral sclerosis (PLS), Creutzfeldt-Jakob disease, primary progressive aphasia, progressive supranuclear palsy, retinitis pigmentosa, macular degeneration, and cognitive deficiency.

As used herein, a “delivery vehicle” or “carrier” means an element capable of carrying any type of cargo, such as small molecules, imaging agents, proteins, peptides, etc. For example, a delivery vehicle can be a polymer, nanoparticle or peptide carrier, and used for drugs or other cargo, and that can be attached to an agent for delivery.

As used herein, “small molecule” refers to a low molecular weight organic compound having a molecular weight of less than 2000 Daltons, in some embodiments less than 1000 Daltons, and in still other embodiments less than 500 Daltons or less. A small molecule is typically between about 300 and about 700 Daltons. In a typical embodiment, a small molecule for use with the invention binds with high affinity to a protein, nucleic acid molecule, or a polysaccharide and alters the activity or function of the biopolymer to which it binds. Such molecules include, for example, heterocyclic compounds, carboxylic compounds, sterols, amino acids, lipids, and nucleic acids.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, “a” or “an” means at least one, unless clearly indicated otherwise.

The practice of the invention involves, unless otherwise indicated, conventional techniques of molecular biology, molecular pharmacology, microbiology, cell biology and recombinant DNA, which are within the skill of those in the art. See, e.g., Sambrook, Fritsch, and Maniatis, Molecular Cloning: A Laboratory Manual, 2nd edition (1989); Current Protocols In Molecular Biology, (F. M. Ausubel et al. eds., 1987); the series Methods In Enzymology (Academic Press, INC.), PCR 2: A Practical Approach (M. J. McPherson, B. D. Hames and (G. R. Taylor eds., 1995); Animal Cell Culture (R. I. Freshney. Ed., 1987); and Antibodies: A Laboratory Manual (Harlow et al. eds., 1987), all fully incorporated herein by reference.

### Amelioration of Endoplasmic Reticulum Stress Response

The invention provides a method of ameliorating an endoplasmic reticulum (ER) stress response and/or unfolded protein response (UPR) in cells expressing nicotinic acetylcholine receptors (nAChRs). The method comprises contacting a cell expressing nAChRs with an effective amount of a ligand for nAChRs. Typically, the contacting comprises intracellular binding of the ligand to nAChRs in endoplasmic reticulum or cis-Golgi apparatus. In a typical embodiment, the contacting results in attenuating endogenously expressed ATF6 translocation, expression of XBP1, phosphorylation of eukaryotic initiation factor 2α (peIF2α), and/or increased numbers of ER exit sites. These are representative intracellular events triggered in response to ER stress and/or an accumulation of unfolded or misfolded proteins in the cytoplasm. Other examples of cellular events triggered by the ER stress response and/or UPR include, but are not limited to, increased trafficking of known associated proteins as well as other proteins such as growth factors and their receptors, changes in abundance of selected mRNA species, and phosphorylation, abundance, or subcellular compartmentalization of other proteins involved with ER stress and/or the UPR. In some embodiments, amelioration of the ER stress response comprises inhibiting upregulation of CCAAT/Enhancer-Binding Protein Homologous Protein (CHOP) levels in the cells.

In one embodiment, the cells are central nervous system (CNS) neurons. Representative examples of such neurons include, but are not limited to, thalamic, cortical, and substantia nigra (SN) neurons. Also included are motor neurons, including upper and lower motor neurons. The cells may be in vivo, ex vivo or in vitro.

Typically, the ligand is an α4β2* agonist, and may be a full or partial agonist. Ligands acting at other nAChRs are also contemplated, including α6β2* nAChRs. In some embodiments, the ligand is an allosteric modulator. Examples of agonists include: cytisine, varenicline, galanthamine, CP-601927, ABT-089, A-85380, (±)-epibatidine, (−)-nicotine, lobeline, sazetidine-A, and derivatives thereof. The ligand may bind to intracellular nAChRs, plasma membrane nAChRs, or both.

### Identifying Neuroprotective Agents

The invention also provides an assay, or method of screening, for identification of neural pharmacotherapeutic agents. In one embodiment, the method comprises contacting a candidate agent with a cell modified to express an α4β2 nAChR; and measuring an indicator of ER stress. The indicator can be selected from expression and/or translocation in the cell of AFT6, phosphorylation of eIF2α, and expression of XBP1, reduced upregulation of CHOP, and measuring ER exit sites, for example. Other examples of cellular events triggered by the ER stress response and/or UPR include, but are not limited to, increased trafficking of known associated proteins as well as other proteins such as growth factors and their receptors, changes in abundance of selected mRNA species, and phosphorylation, abundance, or subcellular compartmentalization of other proteins involved with ER stress and/or the UPR.

The method of screening can further comprise introduction of a UPR activator, such as tunicamycin. This addition to the method allows for identifying agents that attenuate the stress response to a UPR activator. In one embodiment, the cell is pre-treated with the candidate agent prior to introduction of the UPR activator, to allow time for a potentially cumulative protective effect of the agent.

In one embodiment, the cells comprise a clonal mammalian cell line. The clonal cell line can be a HEK or a clone derived therefrom, Neuro2a or a clone derived therefrom, SH-Sy5Y or a parent clone or a clone derived therefrom. In some embodiments, the cells are derived from embryonic cells or from induced pluripotent stems cells

### Methods of Treating and Preventing Neurodegenerative Disease

In one embodiment, the cells are in a patient having, or at risk of developing, a neurodegenerative disorder. The invention thus provides a method of treating, preventing, or delaying the onset of, a neurodegenerative disorder in a subject. The method comprises administering to the subject an α4β2 agonist. Examples of neurodegenerative disorders include, but are not limited to, Alzheimer's disease, Huntington's disease, spinal muscular atrophy, Parkinson's disease, Alzheimer's disease, amyotrophic lateral sclerosis (ALS), progressive muscular atrophy (PMA), progressive bulbar palsy (PBP), primary lateral sclerosis (PLS), Creutzfeldt-Jakob disease, primary progressive aphasia, progressive supranuclear palsy, retinitis pigmentosa, macular degeneration, and cognitive deficiency.

The administering of the ligand is at an effective amount. Typically, an effective amount of ligand is less than about 10%, less than about 1%, or less than about 0.1% of the amount required to activate nAChRs. The contacting can occur for a range of periods, including, for example, 24 hours, 48 hours, at least two weeks, or in some embodiments, for at least two months or longer.

### Pharmaceutical Compositions

The invention provides neuroprotective molecules, compounds and/or agents that are incorporated into pharmaceutical compositions. Pharmaceutical compositions comprise one or more such compounds and, optionally, a physiologically acceptable carrier. Pharmaceutical compositions within the scope of the present invention may contain other compounds that may be biologically active or inactive. For example, one or more portions of other biologically active molecules may be present, either incorporated into a compound or as a separate compound, within the composition.

While any suitable carrier known to those of ordinary skill in the art may be employed in the pharmaceutical compositions of this invention, the type of carrier will vary depending on the mode of administration. Compositions of the present invention may be formulated for any appropriate manner of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

In addition, the carrier may contain other pharmacologically-acceptable excipients for modifying or maintaining the pH, osmolarity, viscosity, clarity, color, sterility, stability, rate of dissolution, or odor of the formulation. Similarly, the carrier may contain still other pharmacologically-acceptable excipients for modifying or maintaining the stability, rate of dissolution, release, or absorption of the delivered molecule. Such excipients are those substances usually and customarily employed to formulate dosages for parenteral administration in either unit dose or multi-dose form or for direct infusion into the CSF by continuous or periodic infusion from an implanted pump.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

The compositions described herein may be administered as part of a sustained release formulation (i.e., a formulation such as a capsule or sponge that effects a slow release of compound following administration, or other implantable device). Such formulations may generally be prepared using well known technology and administered by, for example, oral, rectal or subcutaneous implantation, or by implantation at the desired target site, such as within a muscle. Sustained-release formulations may contain a peptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

### Therapeutic and Prophylactic Methods

Treatment includes prophylaxis and therapy. Prophylaxis or therapy can be accomplished by a single direct injection at a single time point or multiple time points to a single or multiple sites. Administration can also be nearly simultaneous to multiple sites. Patients or subjects include mammals, such as human, bovine, equine, canine, feline, porcine, and ovine animals. The subject is preferably a human.

Peptides or nucleic acid based drugs (e.g., antisense RNAs, siRNAs, mRNAs) can be delivered to cells via chemical means, biological means, carrier peptides, vectors, or physical delivery systems. Representative chemical means include, but are not limited to, specific chemical substances, including cationic polymers such as polyethylenimine (PEI) and cationic lipids. An example of a biological means of delivery is cell-penetrating peptides (CPPs). An exemplary carrier peptide is transportan. Vectors include plasmids and viruses, or cells. Representative physical delivery systems include, but are not limited to electrically-based systems and those using mechanical force, such as gene guns.

Conditions to be treated include, but are not limited to, a neurodegenerative disease. Neurodegenerative diseases are characterized by a progressive loss of structure and/or function of neurons, glial cells, and/or neural structures. Examples of diseases or conditions to be treated include spinal muscular atrophy (SMA), Parkinson's disease, Alzheimer's disease, Huntington's disease, spinocerebellar ataxia, Friedreich's ataxia, amyotrophic lateral sclerosis (ALS), progressive muscular atrophy (PMA), progressive bulbar palsy (PBP), primary lateral sclerosis (PLS), Creutzfeldt-Jakob disease, primary progressive aphasia, Lewy body disease, retinitis pigmentosa, macular degeneration, and progressive supranuclear palsy.

### Administration and Dosage

The compositions are administered in any suitable manner, optionally as pharmaceutically acceptable salts or with pharmaceutically acceptable carriers. Suitable methods of administering compositions, moieties, and molecules in the context of the present invention to a subject are available, and, although more than one route can be used to administer a composition, a particular route can often provide a more immediate and more effective reaction than another route.

The dose administered to a patient, in the context of the present invention, should be sufficient to effect a beneficial therapeutic response in the patient over time, to delay onset of disease, or to inhibit disease progression. Thus, the composition is administered to a subject in an amount sufficient to alleviate, reduce, and cure or at least partially delay or arrest symptoms and/or complications from the disease. An amount adequate to accomplish this is defined as a “therapeutically effective dose.”

A suitable dose is an amount that, when administered as described herein, is capable of promoting a reduction in symptoms, and preferably at least 10-50% improvement over the basal (i.e., untreated) level. Such therapies should lead to an improved clinical outcome (e.g., more frequent remissions, complete or partial or longer disease-free survival) in patients as compared to untreated patients. In general, for pharmaceutical compositions comprising one or more agents, the amount of each agent present in a dose ranges from about 5 μg to 5 mg per kg of host. Suitable volumes will vary with the size of the patient, but will typically range from about 0.1 mL to about 5 mL. For transdermal administration, one example would be a 0.5 mg patch for a 50 kg person. In one embodiment, the patch is applied daily and removed at night, to mimic a smoker's typical pattern of nicotine use.

Routes and frequency of administration of the therapeutic compositions disclosed herein, as well as dosage, will vary from individual to individual, and may be readily established using standard techniques. In general, the pharmaceutical compositions may be administered, by injection or implantation (e.g., intracutaneous, intratumoral, intramuscular, intraperitoneal, intravenous, intrathecal, epidural or subcutaneous), transdermally, intranasally (e.g., by aspiration), by inhalation, by suppository, or orally. In one example, between 1 and 10 doses may be administered over a 52 week period. Preferably, 6 doses are administered, at intervals of 1 month, and booster administrations may be given periodically thereafter, as indicated. Alternate protocols may be appropriate for individual patients. In one embodiment, 2 intradermal injections of the composition are administered 10 days apart. In another embodiment, a dose is administered daily or once every 2 or 3 days over an extended period, such as weeks or months.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

### Kits

For use in the methods described herein, kits are also within the scope of the invention. Such kits can comprise a carrier, package or container that is compartmentalized to receive one or more containers such as vials, tubes, and the like, each of the container(s) comprising one of the separate elements to be used in the method. For example, the container(s) can comprise an agent or a reagent that is, optionally, detectably labeled. The kit can also include one or more containers for a reporter-means, such as a biotin-binding protein, e.g., avidin or streptavidin, bound to a detectable label, e.g., an enzymatic, florescent, or radioisotope label for use in monitoring the agent and/or reagent.

The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic or non-therapeutic application, and can also indicate directions for either in vivo or in vitro use, such as those described above. Directions and or other information can also be included on an insert which is included with the kit.

### EXAMPLES

The following examples are presented to illustrate the present invention and to assist one of ordinary skill in making and using the same. The examples are not intended in any way to otherwise limit the scope of the invention.

### Example 1

### Pharmacological Chaperoning of Nicotinic Acetylcholine Receptors Reduces the Endoplasmic Reticulum Stress Response

This example demonstrates the first observation that endoplasmic reticulum (ER) stress and the unfolded protein response (UPR) can decrease when a central nervous system drug acts as an intracellular pharmacological chaperone for its classical receptor. Transient expression of α4β2 nicotinic receptors (nAChRs) in Neuro-2a cells induced the nuclear translocation of activating translocation factor 6, part of the UPR. Cells were exposed for 48 hr either to the full agonist nicotine, the partial agonist cytisine, or the competitive antagonist dihydro-β-erythroidine. Also tested were mutant nAChRs, which readily exit the ER. Each of these four manipulations increased ER exit sites and attenuated nuclear ATF6-eGFP translocation. However, no correlation was found among the manipulations for other tested parameters: changes in nAChR stoichiometry ((α4)2(β2)3 vs (α4)3(β2)2), changes in ER and trans-Golgi structure, or degree of nAChR upregulation at the plasma membrane. The four manipulations activated zero to 0.4% of nAChRs, showing that activation of the nAChR channel did not underlie the reduced ER stress. Nicotine also attenuated endogenously expressed ATF6 translocation and phosphorylation of eukaryotic initiation factor 2α (peIF2α) in mouse cortical neurons transfected with α4β2 nAChRs. These results demonstrate that when nicotine accelerates ER export of α4β2 nAChRs, this suppresses ER stress and the UPR. Suppression of a sustained UPR may explain the apparent neuroprotective effect that causes the inverse correlation between a person's history of tobacco use and susceptibility to developing Parkinson's disease. This suggests a novel mechanism for neuroprotection by nicotine.

This Example uses the following abbreviations: ATF6—activating transcription factor 6; DHEβE—dihydro-β-erythroidine; DA—Dopaminergic; eCFP—enhanced cyan fluorescent protein; eGFP—enhanced green fluorescent protein; eIF2α—eukaryotic initiation factor 2 α; ER—endoplasmic reticulum; ERES—endoplasmic reticulum exit sites; FA—fractional area; FP—fluorescent protein; FRET—Förster resonance energy transfer; nAChRs—neuronal nicotinic acetylcholine receptors; N/C ratio—nuclear to cytoplasmic ratio; NFRET—normalized Förster resonance energy transfer; nF—net FRET; PD—Parkinson's disease; PM—plasma membrane; RSE—relative standard error; ROI—region of interest; SBT—spectral bleed through; SePhaChARNS—selective pharmacological chaperoning of acetylcholine receptor number and stoichiometry; TG, TGN—trans-Golgi, trans-Golgi network; TIRFM—total internal reflection fluorescence microscopy; UPR—unfolded protein response; wt—wildtype.

The best-known effect of chronic exposure to nicotine is to upregulate nAChRs in vivo and in vitro via cell-delimited, post-translational mechanisms (Buisson and Bertrand, 2002; Gopalakrishnan et al., 1997; Lester et al., 2009; Nashmi et al., 2007; Peng et al., 1994; Sallette et al., 2005; Schwartz and Kellar, 1983; Srinivasan et al., 2011). Emerging data suggest that selective pharmacological chaperoning of acetylcholine receptor number and stoichiometry (SePhaChARNS) by nicotine underlies nicotine-induced nAChR upregulation (Kuryatov et al., 2005; Lester et al., 2009; Miwa et al., 2011; Sallette et al., 2005; Srinivasan et al., 2011). We hypothesized that SePhaChARNS can also attenuate the UPR and can partly explain the observed neuroprotective effects of nicotine in PD.

To test the ER stress/UPR hypothesis, we employed the rich pharmacology of α4β2 nAChRs. nAChRs may be chaperoned by three distinct classes of α4β2 ligands: full agonists (we chose nicotine itself), partial agonists (we chose cytisine). and antagonists (we chose dihydro-β-erythroidine—DHβE) (Gopalakrishnan et al., 1997; Kishi and Steinbach, 2006; Whiteaker et al., 1998). We added a fourth, non-pharmacological manipulation: expression of a previously described β2_{enhanced-ER-export} mutant subunit (also called β2-DM) known to undergo enhanced ER export (Srinivasan et al., 2011).

Exposure to all four manipulations does decrease the UPR, and this correlates well with increased ER exit sites. The tested manipulations, however, display differential effects at all other stages of receptor stabilization and trafficking. Our results point to the possibility that SePhaChARNS can decrease the UPR by increasing cargo export from the ER and altering the physiology of ER. The data establish that pharmacological chaperoning of a CNS receptor by a drug can decrease ER stress and attenuate the UPR, relevant to the apparent neuroprotective effects of nicotine in PD.

### Materials and Methods.

Reagents.

PfuTurbo Cx Hotstart polymerase was purchased from Agilent Technologies. Mouse Neuro-2a cells (CCL-131) were obtained from American Type Culture Collection (ATCC). Dulbecco's modified Eagle's medium (DMEM) with 4 mM L-glutamine. OptiMEM 1, Leibovitz L-15 imaging medium, Glutamax. Neurobasal medium, lipofectamine 2000 and fetal bovine serum (FBS) were purchased from Invitrogen. neupherin neuron was purchased from Biomol. Expressfect was purchased from Denville Scientific. Poly-D-lysine coated and uncoated 35-mm glass bottom imaging dishes (0.19 mm coverslip thickness) were obtained from MatTek corporation. Nicotine, cytisine and DHβE were purchased from Sigma-Aldrich.

Plasmid Constructs.

Mouse β2-eGFP and β2-mcherry were engineered as previously described (Nashmi et al., 2003). All other plasmids used in this study have been previously described (Srinivasan et al., 2011). ATF6-eGFP was obtained from Dr. Ron Prywes (Columbia University, NY, USA) (Shen et al., 2005).

Cell Culture and Transfections.

Mouse Neuro-2a cells were cultured using standard tissue culture techniques and maintained in 45% DMEM, 45% OptiMEM and 10% FBS. Plasmid concentrations used for transfection were as follows: 500 ng of each nAChR subunit, 75 ng of pCS2-mcherry, 250 ng of Sec24D constructs, 100 ng of pDsRed2-ER, 250 ng of GaIT-eCFP and 500 ng of ATF6-eGFP. 90,000 cells were plated in poly-D-lysine coated 35 mm MatTek glass bottom imaging dishes. The following day, plasmid DNA was mixed with cationic lipids by adding appropriate DNA concentrations to 4 μl of Expressfect transfection reagent in 200 μl DMEM final volume and incubated for 20 min at room temperature to form cationic lipid-DNA complexes. DMEM+DNA-lipid complexes were added to Neuro-2a cells in 1 ml of DMEM+10% FBS and incubated at 37° C. for 4 h. Dishes were rinsed 2× with DMEM, then filled with 3 ml of DMEM+10% FBS and incubated at 37° C. for 48 h. Nicotine, cytisine or DHβE was added at appropriate concentrations during the change of medium following 4 h incubation with Expressfect-plasmid DNA complexes.

Neuronal Transfection and Immunostaining.

Cortical neurons were extracted from day 17 mouse embryos; 150,000 cells were plated in each 35 mm poly-l-lysine-coated glass bottom culture dish in a solution containing Neurobasal, B27, and Glutamax supplemented with 3% equine serum. On day four of culture, neurons were treated with 1 μM cytosine arabinoside. Neurons were maintained via 50% exchange with feeding medium (Neurobasal, B27, and Glutamax) twice per wk. Neurons were transfected five days after plating as follows: 1 μg of each nAChR subunit plasmid was incubated with 20 μg Nupherin Neuron in 400 μl of Neurobasal medium. 10 μL of Lipofectamine 2000 was separately incubated for 15 min in 400 μl Neurobasal medium. The two solutions were combined and incubated for 45 min at room temperature, then applied to cells. After 1 hr incubation, the total medium was replaced with fresh 2 ml medium with or without 0.1 μM nicotine. For immunostaining, cultures were fixed the following day with 4% paraformaldehyde (10 min), permeabilized in 0.02% Tris-buffered saline (TBS)-TritonX (15 min) and blocked in 10% goat serum (30 min). Following 2×TBS washes, the appropriate primary antibody in 1% goat serum was applied overnight at 4° C. The following day cultures were rinsed in TBS, and incubated in 1% goat serum containing secondary antibody (1:5000) (30 min). Cells were rinsed in TBS and imaged immediately.

Antibodies.

Immunostaining employed ATF6 mouse monoclonal (Abcam, 11909, 1:50 dilution), rabbit polyclonal peIF2α and total eIF2α (Cell Signaling Technology, 9721 and 5324, both 1:200 dilution). Alexa 488-labeled secondary antibodies were used (Invitrogen, donkey anti-mouse and goat anti-rabbit).

Patch-Clamp Recording.

Neuro-2a cells were plated on 12 mm glass coverslips within 35 mm plastic bottom cell culture dishes. The following day, cells were transfected with 500 ng α4-eGFP and 500 ng β2-wt nAChR subunits. 24 to 48 hr later, glass coverslips were transferred to dishes on the microscope stage. Green fluorescence was visualized with an upright microscope (BX50WI, Olympus) and UV illumination with appropriate excitation filters. Agonist-induced currents were tested at a holding potential of −65 mV using a focal, relatively rapid drug application system designed to minimize desensitization (Xiao et al., 2009). We applied drugs from the lowest to highest concentration at 3 min intervals to minimize desensitization. Whole-cell patch-clamp recordings were with a MultiClamp 700B amplifier, a 1322 analog-to-digital converter, and pCLAMP 9.2 software (all from Axon Instruments, Molecular Devices). Data were sampled at 10 kHz and filtered at 2 kHz.

The intrapipette solution contained (in mM): 135 potassium gluconate, 5 KCl, 5 EGTA, 0.5 CaCl2, 10 HEPES, 2 Mg-ATP, and 0.1 GTP; the pH was adjusted to 7.2 with Tris base and the osmolarity to 300 mOsm with sucrose. The extracellular solution contained (in mM): 140 NaCl, 5 KCl, 2 CaCl₂, 1 MgCl₂, 10 HEPES, and 10 glucose; pH was adjusted to 7.3 with Tris base. The Nernst potential for Cl⁻ in the intrapipette solution is −82.9 mV. The bath was continuously perfused with extracellular solution (ECS) at room temperature (23±1° C.). The patch electrodes had resistances of 5 to 8 MΩ. The junction potential between the patch pipette and the bath solutions was nulled just before forming a gΩ seal. Series resistance was monitored and compensated by 70-80% throughout recordings. The data were ignored if the series resistance (15-30 MΩ) changed by >20% during the recording session.

ATF6-eGFP Translocation Assay.

An Eclipse C1si laser scanning confocal microscope equipped with a 63×, 1.4 numerical aperture VC Plan Apo oil objective and a 32-anode photomultiplier tube (Nikon) was used. Prior to imaging, cell culture medium was replaced with phenol red free CO₂-independent Leibovitz L-15 medium. All experiments were performed in live cells 48 h after transfection at 37° C. in a stage-mounted culture dish incubator (Warner Instruments). For quantifying ATF6-eGFP translocation, the cell was focused to a plane where the nucleus was most clearly visualized and full emission spectra were acquired following simultaneous excitation of mcherry and eGFP with 488 nm and 561 nm laser lines. Images were unmixed using spectra from cells expressing only α4-mcherryβ2-wt nAChRs or ATF6-eGFP. ROIs were manually demarcated for the nucleus and whole cell from the unmixed α4-mcherryβ2-wt image and then applied to the unmixed ATF6-eGFP image of the same cell. Ratios of integrated densities for nucleus divided by whole cell ATF6-eGFP fluorescence were obtained for each cell.

ER Exit Site (ERES) Quantification.

Neuro-2a cells were co-transfected with 500 ng each of α4-mcherry and β2-wt nAChR subunits and 250 ng of the ERES marker, Sec24D-eGFP. Each cell was focused to a plane where the maximum ERES were visualized. Sequential images of eGFP and mcherry fluorescence were obtained and linearly unmixed. For quantification, ERES regions of interest (ROIs) were demarcated using intensity-based thresholding and counted for each cell using the particle analysis feature in ImageJ version 1.44 or later. The fluorescence intensity was obtained for each ERES body within the cells and average ERES intensity was plotted. Error bars for ERES measurements are ±standard error of mean (SEM) and p values are based on a 2-tailed t test.

Quantification of PM Localized nAChRs.

Details of the quantification method have been previously described (Srinivasan et al., 2011). Briefly, PM fluorescence was extracted as follows: Raw TIRF images were converted to background-subtracted images and the ER fluorescence was thresholded and selected. ER fluorescence was then subtracted from the original image to generate images with PM fluorescence signals. These procedures yielded a dataset of several hundred thousand pixel intensities over the 15-50 cells in each experimental group. Fluorescence intensities are simply the sum of pixel values for either the PM or the ER or the ER+PM (using whole TIRF footprint images). Mean PM fluorescence intensity was derived by dividing the total fluorescence intensity for each experimental group by the number of imaged cells and normalized to intensity of untreated cells. Ratios of fluorescence intensities for the whole TIRF footprint (ER+PM) to ER were used to determine the post-Golgi fraction of receptors. For PM integrated density measurements, rather than plotting SEMs (which would be indistinguishably small on the plots), we have used “error bars” to depict 99% confidence intervals based on a two-tailed t-test. Measurements from drug treated cells were normalized to values of untreated cells imaged on the same day. DHβE TIRF experiments were performed on a different day from nicotine and cytisine experiments.

Quantification of Trans-Golgi+Trans-Golgi Network (TG+TGN), TGN Bodies, and ER Fluorescence.

Neuro-2a cells were transfected with α4-eGFP (500 ng)+β2wt (500 ng)+GaIT-mcherry (250 ng) or pDSred2-ER (100 ng) and cells were incubated for 48 h with appropriate concentrations of nicotine, cytisine or DHβE.

For measuring ER area, imaging was performed with 488 nm TIRF illumination and simultaneous collection of nAChR (eGFP) and ER (DSred2) emission. In ImageJ, background was subtracted, and DSred2-ER fluorescence was thresholded and demarcated for each cell.

For TG+TGN fluorescence experiments, imaging was performed with 488 nm TIRF illumination and simultaneous collection of nAChR (eGFP) and TG+TGN (mCherry) emission. GaIT-mcherry was used to label the TG, TGN (Schaub et al., 2006). Two color fluorescence signals were separated with a dichroic and optical filters aligned in the emission path where green and red emission was separately aligned to the two halves of an EMCCD. Post-acquisition processing of images used Metamorph to demarcate TGN bodies and to extract number, size and intensity statistics.

Pixel-by-Pixel Normalized Förster Resonance Energy Transfer (NFRET) from Sensitized Acceptor Emission.

Methods for pixel-by-pixel NFRET from sensitized acceptor emission have been previously described (Moss et al., 2009; Son et al., 2009; Srinivasan et al., 2011). For NFRET experiments, Neuro-2a cells were transfected with GaIT-eCFP+α4-mcherry+β2-eGFP subunits. GaIT-eCFP was used to label the TG, TGN (Schaub et al., 2006). Cells transfected with α4-mcherryβ2wt or α4wtβ2-eGFP nAChRs were included in every imaging session to control for pixel saturation and spectral bleedthrough. Live cells were imaged using an Eclipse C1si laser scanning confocal microscope. During image acquisition, cells were focused to a plane where the GaIT-eCFP fluorescence was best visualized. Images with emission spectra for eCFP, eGFP and mcherry were acquired in 5-nm bins between 470 and 620 nm. 439, 488 and 561 nm laser lines were used to excite eCFP, eGFP and mcherry respectively. Images were linearly unmixed using reference spectra for eCFP, eGFP and mcherry with emission maxima at 477, 508 and 608 nm. Reference spectra were acquired from Neuro-2a cells transfected with either GaIT-eCFP or α4-mcherryβ2wt or α4wtβ2-eGFP during the same imaging session. Linear unmixing was used to separate all three emission spectra from each image. Linearly unmixed images were compiled into donor spectral bleed-through stacks, acceptor spectral bleed-through stacks, and sample image stacks.

The PixFRET ImageJ plugin was used to determine the eGFP and mcherry spectral bleedthrough (BT) values and to calculate the net FRET (nF) (equation 1) and normalized FRET (NFRET) at each pixel. With the background and bleed-through corrections set, the nF for each pixel as described by equation 1 was calculated and the data were presented as 32-bit images. nF was divided by the square root of eGFP and mcherry intensities (equation 2) to obtain the normalized FRET (NFRET) value at each pixel. FRET normalization was used to control for large differences in fluorophore expression within subcellular regions and between different cells in a dish.

$$\begin{matrix}nF = {I}_{FRET} - {I}_{eGFP} \times {BT}_{eGFP} - {I}_{mcherry} \times {BT}_{mcherry} & ( equation 1 ) \\ NFRET = \frac{{I}_{FRET} - {I}_{eGFP} \times {BT}_{eGFP} - {I}_{mcherry} \times {BT}_{mcherry}}{\sqrt{{I}_{eGFP} \times {I}_{mcherry}}} & ( equation 2 )\end{matrix}$$

where, nF=net FRET, I_{FRET}=mcherry sensitized emission with 488 nm excitation, I_{eGFP}=eGFP emission with 488 nm excitation, BT_{eGFP}=spectral bleedthrough of eGFP emission into mcherry emission spectra at 488 nm excitation, I_{mcherry}=mcherry emission with 561 nm excitation and BT_{mcherry}=spectral bleedthrough of mcherry emission into eGFP emission spectra at 561 nm excitation. I_{FRET}, I_{eGFP} and I_{mcherry} were obtained from Neuro-2a cells transfected with GaIT-eCFP+α4-mcherry+β2-eGFP. BT_{eGFP} and BT_{mcherry} values were obtained from Neuro-2a cells transfected with α4-mcherry+β2wt and α4wt+β2-eGFP respectively.

Histograms of NFRET (x-axis) versus number of pixels (y-axis) of each imaged cell were compiled for either the whole cell sections or the TG, TGN ROIs, demarcated by intensity-based thresholding of GaIT-eCFP fluorescence. Frequency histograms obtained in this way (omitting values of zero) were merged for all cells in each experimental group and fitted to two Gaussian components such that a minimum goodness of fit (R²) value of 0.995 was achieved. Ratios of area under the curve for the two Gaussian components with high and low mean NFRET values were used to determine changes in receptor stoichiometry between experimental groups: NFRET histograms were fitted to Gaussian components, yielding the total pixel area under either the A1 (low mean NFRET fit) or A2 (high mean NFRET fit) components. For quantitative measurements of stoichiometry, we compared the fractional area (FA) for the low NFRET component in each case as given by the following equation:

$$FA = \frac{A 1}{( A 1 + A 2 )} \qquad ( equation 3 )$$

where, FA=fractional area, A1=area under the curve for low mean NFRET Gaussian component and A2=area under the curve for high mean NFRET Gaussian component.

Due to variability resulting from cell passage number, we imaged untreated control cells on the same day to compare and evaluate the effects of drug exposure on stoichiometry.

### Results

Each of the Four Manipulations Inhibits the Nuclear Translocation of ATF6.

We hypothesized that nAChR expression and upregulation can affect the ER stress/unfolded protein response (UPR) (Srinivasan et al., 2011). During the UPR, ATF6 translocates from the ER to the Golgi and is cleaved by site 1 and site 2 proteases (SP1 and SP2) to release an N-terminal peptide. The N-terminal ATF6 peptide translocates to the nucleus and initiates transcription of several target UPR genes that enhance ER function (Bommiasamy et al., 2009; Maiuolo et al., 2011; Matsushita et al., 2002).

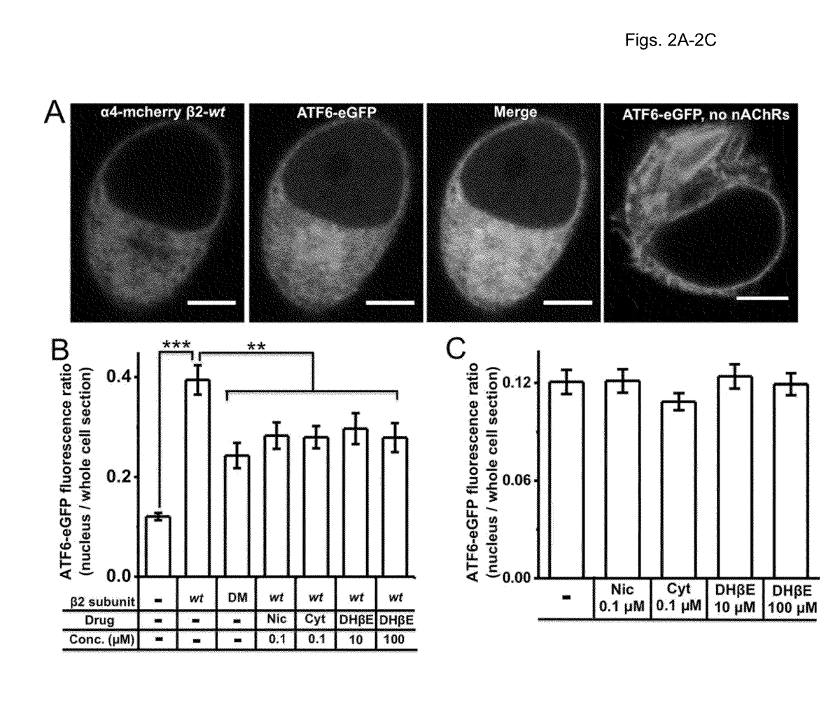

To test the effect of nAChR expression on nuclear ATF6 translocation, we utilized a previously reported ATF6 construct with an N-terminal eGFP fusion (ATF6-eGFP) (Shen and Prywes, 2005; Shen et al., 2005). Neuro-2a cells were transfected with either ATF6-eGFP alone or ATF6-eGFP and α4-mcherryβ2-wt nAChRs (FIG. 2A). We found that co-expression of α4-mcherryβ2-wt nAChRs increased nuclear ATF6-eGFP translocation (FIGS. 2A and B). To evaluate if the observed increase in ATF6-eGFP translocation to the nucleus was an artifact of DNA transfection or protein overexpression, Neuro-2a cells were co-transfected with ATF6-eGFP and α4-mcherry, paired with either β2-wt or a previously described β2_{enhanced-ER-export} mutant subunit (also called β2-DM) known to undergo enhanced ER export (Srinivasan et al., 2011). Compared to α4-mcherryβ2-wt nAChRs, expression of α4-mcherryβ2_{enhanced-ER-export} mutants caused a 40% reduction in the ratio of nuclear to whole cell ATF6-eGFP fluorescence in the absence of nicotine (FIG. 2B).

In parallel experiments, Neuro-2a cells co-expressing ATF6-eGFP and α4-mcherryβ2-wt nAChRs were treated with nicotine, cytisine (0.1 μM, 48 h) or DHβE (10 or 100 μM, 48 h). Nicotine and cytisine caused ˜25% reduction in the ratio of nuclear to whole cell ATF6-eGFP fluorescence (FIG. 2B) and both concentrations of DHβE reduced the ratio by ˜23% (FIG. 2B). None of the tested ligands affected ATF6-eGFP translocation in the absence of nAChR co-expression (FIG. 2C).

Nicotine Inhibits Endogenous ATF6 Translocation and Prevents of Eukaryotic Initiation Factor 2α (eIF2α) Phosphorylation in Primary Mouse Cortical Neurons.

We sought to study the effects of nicotine on the UPR in primary neurons in culture. Cells were transfected with α4-mcherryβ2wt nAChRs and separately immunostained for endogenous ATF6, total eIF2α or phosphorylated eIF2α (peIF2α).

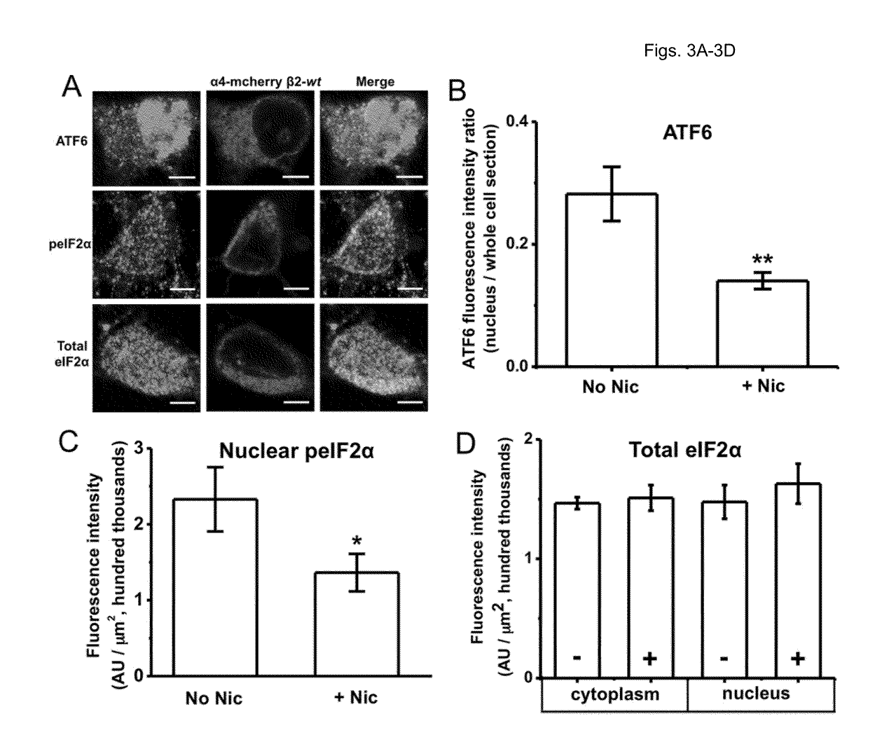

In the absence of nicotine, 56% of α4-mcherryβ2wt-expressing cells showed nuclear ATF6 localization (FIG. 3A), which reduced to 14% following 24 hr treatment with 0.1 μM nicotine. Compared to untreated cells, nicotine caused a significant 50% reduction in the nucleus to whole cell ATF6 fluorescence ratio (FIG. 3B). peIF2α and total eIF2α localized to the nucleus and cytoplasm of all α4-mcherryβ2wt-expressing neurons (FIG. 3A). 24 h exposure to 0.1 μM nicotine showed a trend towards reduction in whole cell and cytoplasmic peIF2α fluorescence (p=0.06), while nuclear peIF2α was significantly reduced compared to untreated controls (FIG. 3C). Nicotine did not affect total eIF2α fluorescence within the cytoplasm or nucleus of neurons (FIG. 3D), indicating specific nicotine-induced inhibition of eIF2α phosphorylation.

Each of the Four Manipulations Increases ER Exit Site Formation.

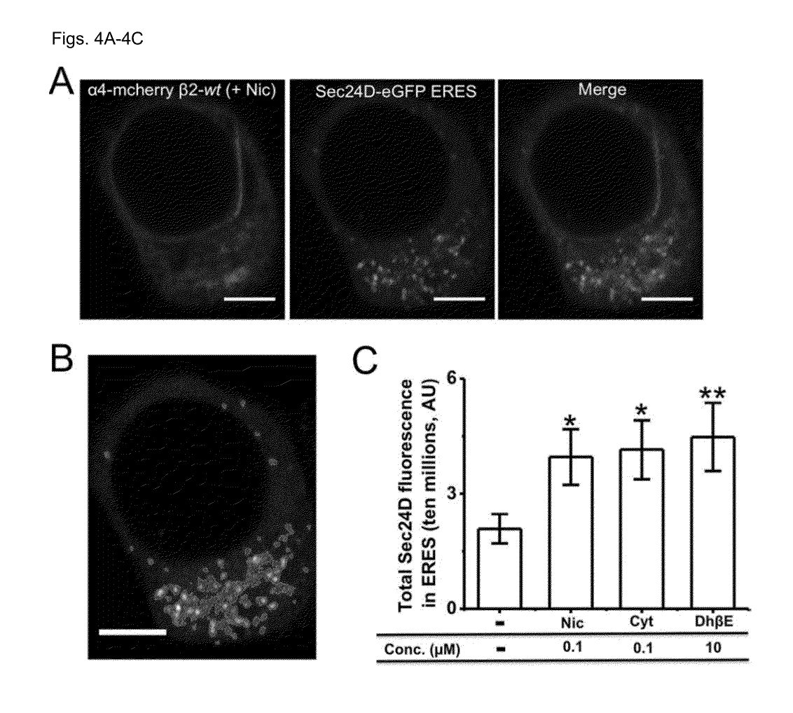

To understand the mechanistic basis for the effects of nicotinic ligands on the UPR, we studied the effects of each ligand on ER exit site (ERES) formation. Neuro-2a cells were transfected with α4-mcherryβ2-wt nAChRs and Sec24D-eGFP was used as a marker to quantify ERES (FIG. 4A). We quantified fluorescence from condensed Sec24D-eGFP ERES structures that contributed to the upper third of total Sec24D-eGFP fluorescence in each cell (FIG. 4B). All three nicotinic ligands caused a significant 1.5 to 2-fold increase in ERES fluorescence compared to untreated control cells (FIG. 4C). These data imply that ligand-induced UPR inhibition arises, at least in part, from increased cargo exit from the ER. Previous data show that replacement of the β2 subunit by the β2_{enhanced-ER-export} subunit also enhances the formation of ERES (Srinivasan et al., 2011).

the Manipulations have Diverse Effects on the Morphology of the ER and Trans-Golgi Network.

Total internal reflection fluorescence microscopy (TIRFM) can be used to determine the subcellular localization of FP-labeled proteins at the cellular periphery (Richards et al., 2011). These experiments have shown that a majority of wt α4β2 receptors localize to the ER (Srinivasan et al., 2011). Here, we used TIRF microscopy to quantify the effect of ligand-induced nAChR upregulation on the ER and trans-Golgi network (TGN) architecture. For quantification, we utilized fluorescence from specific FP-tagged markers of the ER (DSred2-ER) or the TGN (GaIT-mcherry) in the presence of co-expressed α4β2 nAChRs. Thus, the results described here assess the effect of the ligand-α4β2 interaction on general ER and TGN morphology.

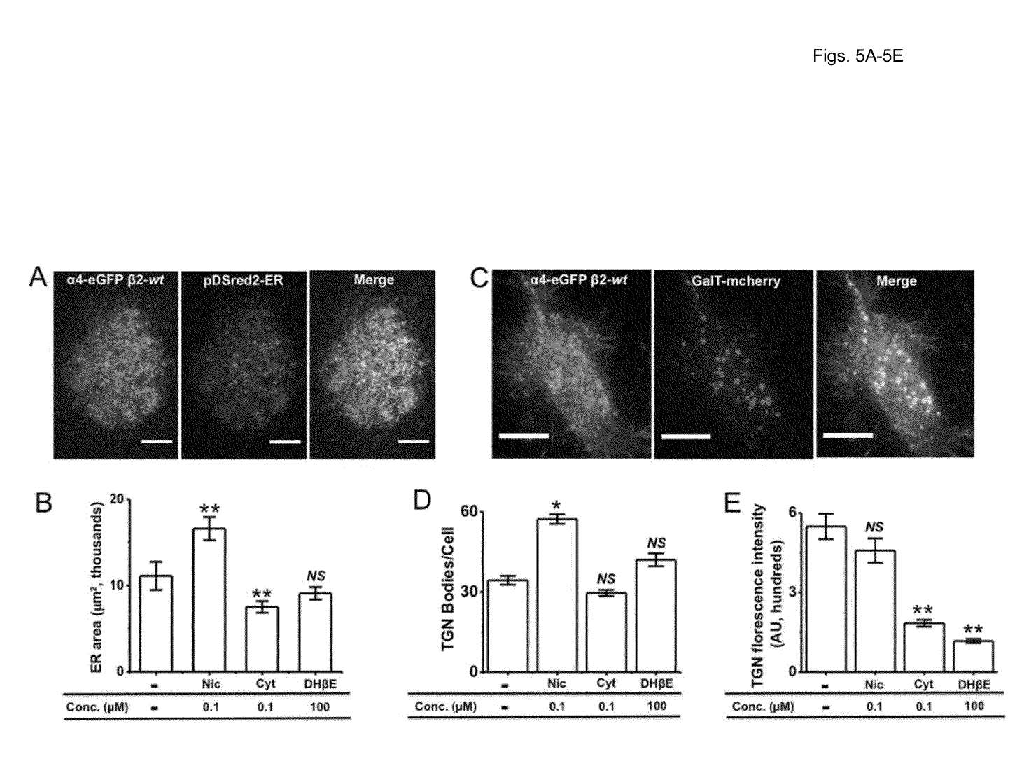

In a first set of TIRFM experiments, Neuro-2a cells were co-transfected with α4-eGFPβ2-wt nAChRs and DSred2-ER (ER marker). Expressed α4-eGFPβ2-wt nAChRs co-localized almost completely with DSred2-ER fluorescence (FIG. 5A). We utilized DSred2-ER fluorescence to demarcate and quantify the average area of peripheral ER. Compared to untreated controls, nicotine exposure caused a 1.7-fold increase in the average ER area, while cytisine caused a significant ˜1.4-fold reduction and DHβE did not significantly affect the ER area (FIG. 5B).

In a second set of TIRFM experiments, Neuro-2a cells were co-transfected with α4-eGFPβ2-wt and GaIT-mcherry to demarcate the TGN (FIG. 5C). In this case, we quantified the average number and intensity of GaIT-mcherry labeled TGN bodies. Nicotine caused a significant ˜2-fold increase in the average number of TGN bodies per cell when compared with untreated cells, while cytisine and DHβE failed to significantly affect the number of TGN bodies (FIG. 5D). In addition, nicotine exposure did not affect the TGN intensity, while cytisine and DHβE significantly reduced the average intensity of TGN bodies (FIG. 5E). These data show that receptor chaperoning by nicotine, cytisine and DHβE alters the morphology of ER and the TGN in a ligand-dependent manner.

The Four Manipulations have Diverse Effects on nAChR Stoichiometry.

We tested the effects of the three ligands on the stoichiometry ((α4)₂(β2)₃ vs (α4)₃(β2)₂) of assembled pentameric nAChRs, primarily in organelles. These experiments implement a Förster resonance energy transfer (FRET) method for monitoring nAChR stoichiometry (see Methods). In measurements of normalized FRET (NFRET), the fractional area (FA) of the fitted low NFRET component increases monotonically with the ratio of (α4)₂(β2)₃ to total ((α4)₂(β2)₃ and (α4)₃(β2)₂) nAChRs.

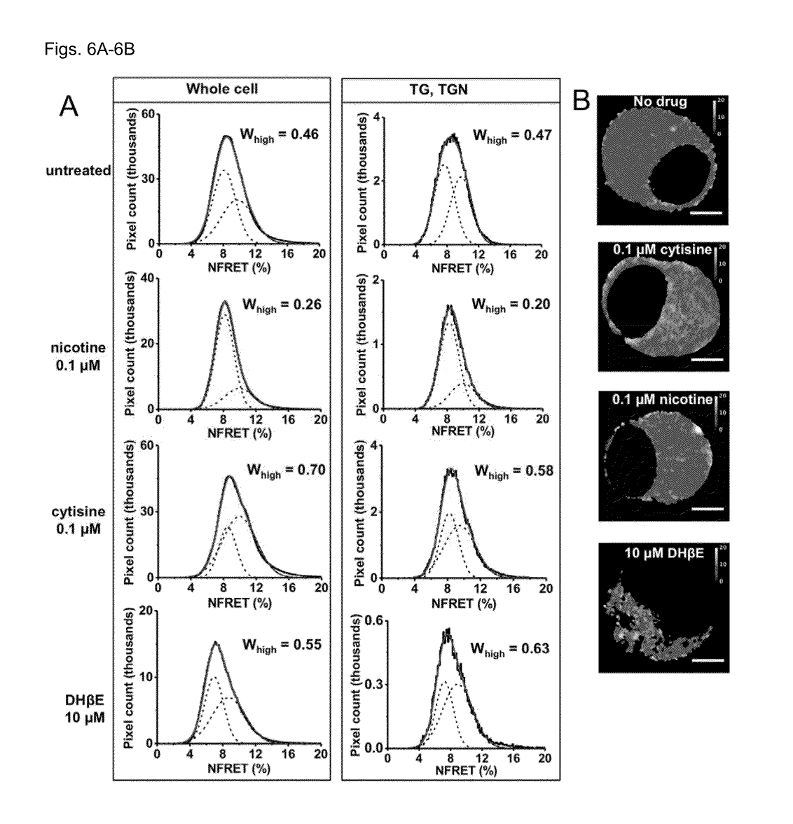

Neuro-2a cells were transfected with α4-mcherry, β2-eGFP and a trans-Golgi+trans-Golgi network (TG+TGN) marker, GaIT-eCFP. Whole cell and TG+TGN FAs for untreated cells were 0.54 and 0.53 respectively, while in nicotine treated cells, whole cell and TG+TGN FAs increased to 0.74 and 0.80 respectively (FIG. 6A). In contrast to nicotine, cytisine-treated cells showed a net reduction in the FA for the whole cell (FA=0.30) and TG+TGN (FA=0.42) when compared to untreated controls (FIG. 6A). DHβE did not affect nAChR stoichiometry at concentrations ranging from 0.1 to 1 μM, but 10 μM DHβE decreased the whole cell FA to 0.45 and reduced the TG+TGN FA to 0.37 (FIG. 6A). It should be noted that when compared with untreated, nicotine- or cytisine-treated cells, we observed a 3- to 5-fold reduction in the number of NFRET positive pixels for the whole cell and TG+TGN following treatment with either 10 or 100 μM DHβE (FIG. 6B).

These results demonstrate that nicotine and cytisine stabilize the assembly of (α4)₂(β2)₃ and (α4)₃(β2)₂ receptors respectively, while DHβE weakly favors the assembly of (α4)₃(β2)₂ receptors. Previous data show that replacement of the β2 subunit by the β2_{enhanced-ER-export} subunit also stabilizes the (α4)₂(β2)₃ stoichiometry (Srinivasan et al., 2011). All four manipulations produce changes in stoichiometry prior to export of receptors from the ER to the Golgi.

The Tested Agonist Concentrations Minimally Activate α4β2 nAChRs.

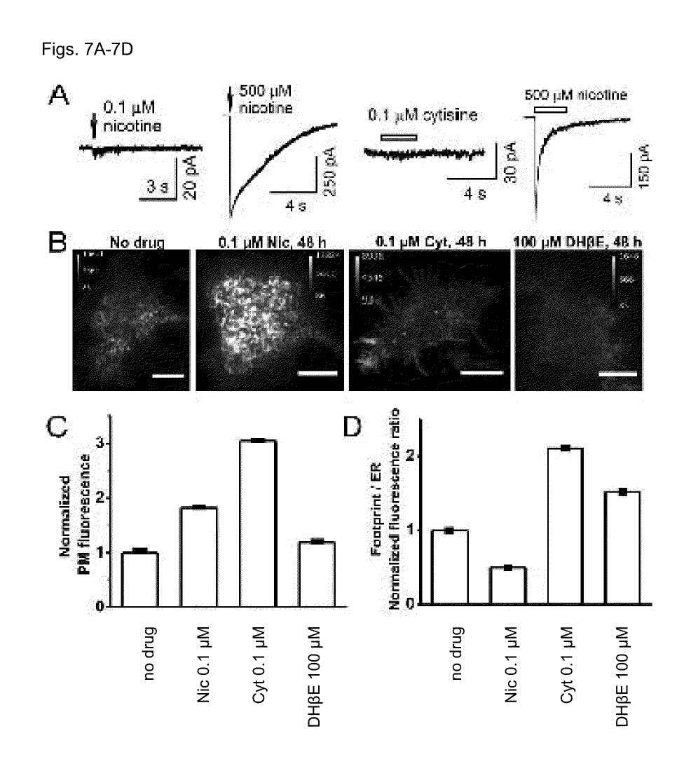

Since the observed effects of nicotinic agonists on the cellular secretory pathway occurred at 0.1 μM, we sought to determine whether, and to what extent, this concentration of nicotine and cytisine activates α4β2 nAChRs. Whole cell electrophysiological responses to focal nicotine and cytisine puffs were measured in Neuro-2a cells transiently expressing α4-eGFPβ2-wt nAChRs. FIG. 7A shows representative traces with 0.1 μM nicotine or cytisine. Measured current amplitudes were normalized to the maximum current amplitude obtained by puffing 500 μM nicotine (FIG. 7A). At 0.1 μM, we observed nicotine evoked currents that were 0.44±0.07% (n=4) of maximum currents induced by 500 μM nicotine. In the case of cytisine, 0.1 μM activated <0.1% (n=7) of maximum currents induced by 500 μM nicotine. These data indicate that the effects of nicotine and cytisine on nAChR stoichiometry are likely independent of nAChR activation and ion flux.

The Four Manipulations have Diverse Effects on Upregulation of PM nAChRs.

We utilized TIRFM to quantify the effects of nicotine, cytisine and DHβE on nAChRs at the PM of Neuro-2a cells. Pixel-by-pixel TIRFM quantification is a direct measure of PM localized receptors (Srinivasan et al., 2011). In addition, ratios of whole footprint to ER localized fluorescent nAChRs (footprint/ER ratio) provide a quantitative estimate of nAChRs in the PM versus the peripheral ER (Srinivasan et al., 2011). For these experiments, cells were transfected with α4-eGFPβ2-wt and α4-eGFP fluorescence was used to quantify nAChRs. FIG. 7B shows representative TIRF images of nAChR fluorescence following 48 h exposure to each drug.

When compared to untreated controls, nicotine and cytisine respectively caused 2- and 3-fold increases, while DHβE caused a small, but significant increase in PM-localized nAChRs (FIG. 7C). The footprint/ER ratio reduced by ˜2-fold following nicotine treatment, while cytisine and DHβE-treated cells showed a ˜1.5-fold increase in the footprint/ER ratio compared to untreated cells. (FIG. 7D). Previous data show that replacement of the β2 subunit by the β2_{enhanced-ER-export} subunit also markedly increases the footprint/ER ratio (Srinivasan et al., 2011).

### Discussion

The data establish, for the first time, that pharmacological chaperoning of a CNS receptor by a drug can decrease ER stress and attenuate the UPR. Parkinson's disease (PD) is associated with increased ER stress/unfolded protein responses (UPR) in dopaminergic (DA) neurons. Upon persistent activation, the UPR can cause apoptosis (Hoozemans et al., 2007; Silva et al., 2005). Because nicotine is potentially neuroprotective in PD (Hernan et al., 2002; Lester et al., 2009; Quik et al., 2011), we assessed the subcellular effects of pharmacological chaperoning by nicotinic ligands. Table 1 summarizes data obtained for the four manipulations—three exemplar nicotinic ligands, and nAChRs containing mutant β2_{enhanced-ER-export}. All four manipulations suppress ATF6 translocation, which serves as a key sensor for ER stress and the UPR (Hetz and Glimcher, 2009; Maiuolo et al., 2011; Ron and Walter, 2007); but the only other consistent phenomenon is the increase in condensed ERES fluorescence. Therefore we suggest that increased ER exit of nAChRs underlies the suppression of ATF6 translocation.

Table 1.

The four manipulations all reduce ATF6 translocation and increase condensed ERES, but have diverse effects on other aspects of α4β2 stoichiometry, organelle structure, and PM upregulation. Numbers indicate fold-change from untreated cells. Arrows (↑ or ↓) indicate the fold increase or decrease from observed values in untreated cells. ND=not done. Some of the data for β2-DM are from Srinivasan et al, J. Gen. Physiol. 2011 January; 137(1):59-79.

| Tested parameter | Nicotine | Cytisine | DHβE | β2-DM |
|---|---|---|---|---|
| ATF6 (nucleus: | ↓1.4 | ↓1.4 | ↓1.4 | ↓1.6 |
| whole cell) | | | | |
| Condensed ERES | ↑1.9 | ↑2 | ↑2.1 | ↑2 |
| Peripheral ER area | ↑1.8 | ↓1.5 | ↓1.2 | ND |
| nAChR footprint/ | ↓2 | ↑2 | ↑1.5 | ↑2.3 |
| ER ratio | | | | |
| Number of TGN bodies | ↑2 | no effect | no effect | ND |
| TGN intensity | no effect | ↓3 | ↓3 | ND |
| Major stoichiometry | (α4)₂(β2)₃ | (α4)₃(β2)₂ | (α4)₃(β2)₂ | (α4)₂(β2)₃ |
| PM nAChRs | ↑2 | ↑3 | ↑1.2 | ↑2.5 |

DA neurons show robust expression of several nAChR subtypes (Champtiaux et al., 2003), placing these nAChRs in a key position to influence ER stress by influencing ER exit. In the present experiments, ER stress was caused by expression of the α4β2 nAChRs themselves (FIGS. 2A and 2B); but it is plausible to suggest that increasing the ER exit of nAChRs could re-organize the COPII vesicle population and relieve ER stress caused by other environmental or genetic factors. For instance, dopaminergic toxins, or expression of an α-synuclein linked to Parkinson's disease, induce ER stress in catecholaminergic cell lines and in cultured dopaminergic neurons (Holtz and O'Malley, 2003; Ryu et al., 2002; Thayanidhi et al., 2010).

Several points suggest that the enhanced ATF6 translocation was not related to mere non-specific overexpression. (1) Transient expression of ATF6-eGFP alone did not cause ATF6 translocation to the nucleus (FIG. 2B), and nicotinic ligands did not influence ATF6 localization (FIG. 2C). (2) nAChRs containing mutant enhanced-ER-export β2 subunits prevented ATF6 translocation in the absence of nicotine (FIG. 2B). (3) Our experiments utilized a well-characterized expression system in Neuro-2a cells, which express membrane proteins at rather lower levels compared to the more commonly studied HEK293 cells (Moss et al., 2009). Likewise, neurons typically express transfected genes at only modest levels.

Nicotine also inhibited ATF6 translocation in primary mouse cortical neurons transfected with α4β2 nAChRs (FIGS. 3A and 3B). Thus, the observed effects of nicotinic ligands on ATF6 localization occur in at least two cell types (neurons and a neuroblastoma cell line); these effects do not require ATF6 overexpression or the use of a non-native promoter to drive ATF6 gene expression. During the UPR, PKR-like ER-localized eIF2α kinase (PERK) induces eIF2α phosphorylation, resulting in a global inhibition of protein translation (Harding et al., 2000). A sustained increase in phosphorylated eIF2α can cause C/EBP-homologous protein (CHOP)-mediated apoptosis (Galehdar et al., 2010; Harding et al., 2000; Scheuner et al., 2001). Previous reports have described eIF2α in the nucleus, which may function to regulate transcriptional and translational processes (DuRose et al., 2009; Goldstein et al., 1999; Tejada et al., 2009). We found that nicotine exposure significantly inhibited nuclear eIF2α phosphorylation in α4β2 overexpressing neurons without affecting the expression of total eIF2α (FIGS. 3C and 3D). Nicotine also caused a near significant reduction of phosphorylated eIF2α in the cytoplasm (p=0.06). Together these data indicate that nicotinic ligands can inhibit the UPR via their interaction with nAChRs.

ERES formation is generally linked to some aspects of ER function. Chronic exposure to nicotine, cytisine and DHβE increased the formation of ERES by ˜2-fold in the presence of co-expressed nAChRs (FIG. 4C). In the context of PD neuroprotection, dopaminergic neurons exposed to chronic nicotine would increase the formation of ERES in a nAChR-dependent manner. We suggest that these events can cause a generalized exit of cargo from the ER, resulting in reduced signals to sensors of ER stress

The specification of nAChR stoichiometry is a basic aspect of nAChR assembly that occurs before ER exit. Of the four manipulations tested, two (nicotine and the inclusion of the β2_{export-ER-export} subunit) preferentially stabilize receptors with (α4)₂(β2)₃ stoichiometry, while cytisine and DHβE preferentially stabilize the assembly of (α4)₃(β2)₂ receptors (FIG. 5). Cytisine and DHβE bind strongly at the α4-α4 interface which exists only in the (α4)₃(β2)₉ nAChR, providing the possible structural basis for the preferential stabilization of this stoichiometry (Mazzaferro et al., 2011). Interestingly, both types of stabilization increased additional ERES and suppressed ATF6 translocation. Apparently the nature of the subunit in the fifth or “auxiliary” position is not crucial for interactions with the COPII complex or for ER exit. Since nicotine increased, while cytisine reduced, the area of peripheral ER even though both decreased the UPR (FIG. 5B), the suppression of ATF6 translocation is apparently not sufficient to suppress ER proliferation when triggered by nAChRs, although the suppression of ATF6 translocation is sufficient to suppress ER proliferation when expression of a tail-anchored protein is sensed within the lipid bilayer (Maiuolo et al., 2011).

Events downstream from Golgi exit are not thought to markedly influence the UPR, and these events vary among the manipulations. Nicotine increased the number of TGN bodies per cell, while cytisine and DHβE reduced the average intensity of the TGN bodies (FIGS. 5D and 5E). Nicotine caused an ˜2-fold increase in PM localized nAChRs and a reduction in the footprint/ER integrated density ratio (FIGS. 7C and 7D). By contrast, cytisine treatment resulted in a ˜3.5-fold upregulation of PM receptors and caused ˜2-fold increase in the footprint/ER, while DHβE exposure led to no marked effects on these parameters (FIGS. 7C and 7D). These observations may indicate that the two nAChR stoichiometries stabilized by nicotinic ligands utilize distinct mechanisms for trafficking from Golgi to PM, via differential interactions between the subunits' M3-M4 loops and cytoskeletal or trafficking proteins (Kabbani et al., 2007; Xu et al., 2006).

Nicotine may also regulate nAChR interactions with the ubiquitin-proteasome system (Rezvani et al., 2010; Rezvani et al., 2007). The present study shows reduction of ER stress at nicotine concentrations 500-fold lower than those required to suppress UPR pathways in tunicamycin-treated PC12 cells, where direct actions of nicotine with the ubiquitin-proteasome system were suggested (Sasaya et al., 2008). PC12 cells lack α4β2 nAChRs but express lower-affinity α3β4 nAChRs, which may be subject to chaperoning at relatively high nicotine concentrations.

None of the four tested manipulations produce appreciable nAChR activation, ruling out mechanisms in which ion flux associated with receptor activation affects intracellular signaling cascades, receptor assembly, stoichiometry, and its consequent effects on the UPR. Our electrophysiological experiments confirm, in the transfected Neuro-2a cells under study, that 0.1 μM nicotine or cytisine respectively activate only 0.4% or <0.1% of the total receptor population (FIG. 7A). Moreover, nicotine exposure used in our experiments likely desensitizes α4β2 nAChRs. Furthermore DHβE activates no receptors; and the mutant α4β2_{enhanced-ER-export} receptors also display no constitutive activity. These data agree with previous studies showing that upregulation is independent of surface nAChR activation (Corringer et al., 2006; Sallette et al., 2005). Although these ligand concentrations activate no nAChRs when applied for a few seconds, these concentrations exceed, by factors of 10 to 100, the equilibrium binding dissociation constant measured by incubations of minutes to hours (Gopalakrishnan et al., 1997; Warpman et al., 1998; Whiteaker et al., 1998). Evidently these ligand concentrations stabilize the formation of fully assembled ligand-receptor complexes which can interact with ER-associated export machinery more readily than with ER-associated degradation.

PD becomes clinically apparent after a degenerative process has operated for a decade or longer. The apparent neuroprotective effects of smoking also begin decades before the clinical diagnosis (Ritz et al., 2007). Therefore, nicotine may exert a cumulative protective effect(s) which counteracts the cumulative degenerative mechanism(s). The data establish that pharmacological chaperoning of a CNS receptor by a drug can decrease ER stress and attenuate the UPR, and it is plausible to suggest that this mechanism exerts the required cumulative protective effect. Reductions in ER stress can occur without activating PM nAChRs, suggesting a therapeutic strategy for neuroprotection without the potential for abuse. We do not yet know whether similar reductions in ER stress and UPR occur when nicotine interacts intracellularly with AChRs at endogenous levels in the neurons of intact brains.

References Cited in Example 1:

The references cited in Example 1 are listed in Srinivasan et al., Mol Pharmacol. 2012 June; 81(6):759-69, the entire contents of which are incorporated herein by reference. Also incorporated herein by reference are the entire contents of Srinivasan et al., J. Gen. Physiol. 2011 January; 137(1):59-79.

### Example 2

### Psychiatric Drugs Bind to Classical Targets within Early Exocytotic Pathways: Therapeutic Effects

The classical targets for antipsychotic and antidepressant drugs are G protein-coupled receptors and neurotransmitter transporters, respectively. Full therapeutic actions of these drugs require several weeks. This Example shows how therapeutic effects may eventually accrue after existing therapeutic ligands bind to these classical targets, not on the plasma membrane but rather within endoplasmic reticulum (ER) and cis-Golgi. Consequences of such binding may include pharmacological “chaperoning”: the nascent drug targets are stabilized against degradation and can therefore exit the ER more readily. Another effect may be “matchmaking”: hetero- and homodimers of the target form and can more readily exit the ER. Summarizing recent data for nicotinic receptors, we explain how such effects could lead to reduced ER stress and to a decreased unfolded protein response, including changes in gene activation and protein synthesis. In effects not directly related to cellular stress, “escorting” would allow increased ER exit and trafficking of known associated proteins, as well as other proteins such as growth factors and their receptors, producing both cell-autonomous and non cell-autonomous effects. Axonal transport of relevant proteins may underlie the several weeks required for full therapy. In contrast, the antidepressant effects of ketamine and other NMDA receptor ligands, which occur within <2 h, could arise from dendritically localized intracellular binding, followed by chaperoning, matchmaking, escorting, and reduced ER stress. Thus, the effects of intracellular binding extend beyond proteostasis of the targets themselves and involve pathways distinct from ion channel and G protein activation. The Example describes experimental tests and notes pathophysiological correlates.

What events take place during the two to three weeks required for the full therapeutic actions of an antidepressant or antipsychotic drug? Most workers agree that a process(es) is activated long after the few seconds required for the drug-receptor interaction to attain steady state at the plasma membrane. Signal transduction cascades, maintained for several weeks, are thought to be involved. Among the postulated downstream mechanisms are gene activation and neurogenesis. A satisfactory mechanistic picture would also explain the more rapid antidepressant effects of ketamine and other NMDA glutamate receptor blockers.

The new therapeutic hypotheses reviewed here continue to focus on the classical targets. For the serotonin-selective reuptake inhibitors (SSRIs), the targets are the eponymous serotonin transporter (SERT) and, to a lesser extent, the norepinephrine transporter (NET). For antipsychotic drugs, the dopamine D2/D3 and serotonin 5-HT2A G protein-coupled receptors (GPCRs) comprise the classical targets. Recently described antidepressant effects of ketamine and related compounds occur within 2 h; the target is the NMDA receptor. But in the new hypotheses, the targets are in a novel location: the ER and cis-Golgi, where they are being synthesized and glycosylated (See Supplementary Figure in Lester, et al., Biol Psychiatry. 2012 Dec. 1; 72(11):907-15).

Intracellular actions of psychiatric drugs are not a novel concept. For decades, we have assumed that valproate and Li⁺ act intracellularly, primarily because no high-affinity plasma membrane binding sites have been identified. The sigma-1 receptor, originally thought to be an opioid receptor, presents another relevant example, because it is an intracellular chaperone protein that participates in ER stress signaling (1). The numbers in parentheses throughout Example 2 indicate references that are listed in Lester, et al., Biol Psychiatry. 2012 Dec. 1; 72(11):907-15.

We will not soon have full pathophysiological information about schizophrenia, bipolar disease, or depression. Many therapeutic drugs have been analyzed in the absence of detailed pathophysiology about their target disease. This essay proceeds similarly, but we comment briefly on pathophysiology in the final section.

### Statement of the Hypotheses

Antidepressants and antipsychotic drugs are able to bind intracellularly, in the ER and cis-Golgi, to their nascent receptors. There are several possible sequelae. (1) The target protein achieves a stable state that resists ER associated degradation (ERAD) and/or ER retrieval. This is pharmacological “chaperoning”. (2) In some cases, the intracellular binding enhances assembly or dimerization of the target, providing further stability. This is “matchmaking”. (3) These processes increase the ER exit rate of the receptors. (4) The increased ER exit suppresses one or more arms of ER stress and the unfolded protein response (UPR), improving neuronal function in a cell-autonomous fashion. (5) The increased ER exit allows the targets to co-traffic more effectively with candidate beneficial secreted proteins such as neurotrophins; these can then act in a non-cell autonomous fashion. This is “escorting”. (6) The binding of ligand within the ER can disrupt endogenous trafficking of either the target or a co-trafficked protein. This could be termed “abduction”. Note that (5) and (6) emphasize the molecules “escorted” or “abducted” by the drug-target complex, not the drug targets themselves.

Thus the hypothesis states that the therapeutic effects of psychiatric drugs occur via “inside out” signal transduction beginning in the ER. This contrasts with the usual assumption that therapeutic drugs act “outside-in”, via the membrane-localized drug-receptor interaction. Importantly, we will show how downstream effects can extend beyond proteostasis (2) of the targets themselves.

### Pharmacological Chaperoning and Matchmaking

Our laboratory's work focuses on explaining the molecular, cellular, and circuit-based instantiation of such terms as “plasticity” and “adaptation” during chronic exposure to nicotine. At the subcellular level, nicotine acts as a pharmacological chaperone for α4β2 nicotinic receptors (nAChRs) (3-5). A pharmacological chaperone is a small molecule that stabilizes a protein by binding, as either a substrate, agonist, antagonist, or allosteric modulator, at a physiologically relevant site on the target protein—but the binding primarily occurs within an organelle, and usually during biosynthesis and trafficking of the target protein. A pharmacological chaperone is not a chaperone protein, although in some cases the effects might be similar. We are beginning to understand how pharmacological chaperoning by nicotine underlies both some initial events of nicotine addiction and some apparent neuroprotective actions of nicotine in Parkinson's disease (6).

Nicotinic Ligands, Acting on nAChR, Modify the Unfolded Protein Response

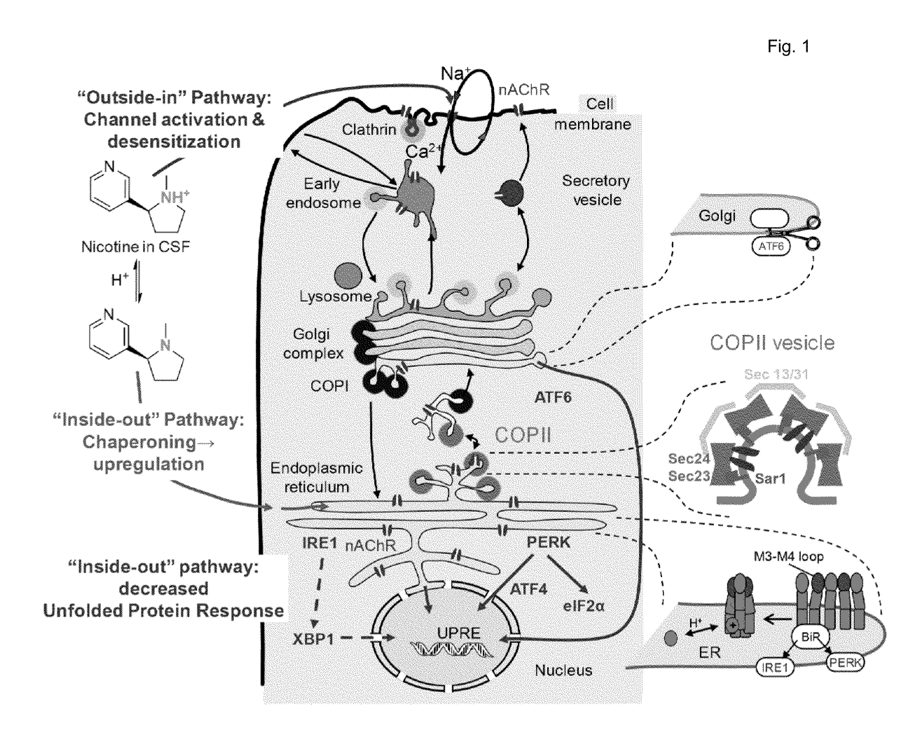

The unfolded protein response (UPR) is a homeostatic mechanism that fine-tunes the cell in response to the demands of newly synthesized proteins that enter the ER. In mammalian cells (See FIG. 1). The unfolded protein response is thought to become activated when an ER-resident chaperone, immunoglobulin binding protein (BiP) (also known as 78 kDa glucose-regulated protein GRP-78, or heat shock 70 kDa protein 5 HSPA5), binds to hydrophobic groups on unfolded or partially folded proteins. As a consequence, BiP dissociates from three other proteins in the ER membrane: protein kinase R-like ER-localized eIF2α kinase (PERK), inositol-requiring enzyme 1 (IRE1), and ATF6. Each of these three proteins then activates a pathway resulting in gene activation, in protein synthesis, and in posttranslational modifications. The overall result initially increases the protein processing capacity in the ER, but the PERK-eIF2α pathway eventually decreases the level of translation by membrane-bound ribosomes (7-10).

Many details of the UPR are emerging. During the UPR, some genes are activated while others are repressed. Of particular interest to those studying receptors and transporters, membrane proteins may produce a signal that triggers a UPR, even if they have no lumenal domain (which would eventually become extracellular); and the three pathways may be activated differentially during the lumenal vs transmembrane triggers (11).

When ER stress and the UPR continue for long periods, they appear capable of reducing cell function as well. ER stress occurs in certain dystonia subtypes caused by defective ER-associated proteins (12) and in several examples of inflammation (13). The most extreme outcome is apoptosis. ER stress and the UPR are clearly activated during several neurodegenerative diseases (14, 15).

We review here, for the first time, our recent work indicating that the intracellular interaction between nicotine and nAChRs is sufficient to modulate ER stress and to decrease the UPR. These effects were monitored by the number of Sec24d molecules in condensed endoplasmic reticulum exit sites, by translocation of activating transcription factor 6 (ATF-6), and by phosphorylation of eIF2α. Three ligands tested—the full agonist nicotine, the partial agonist cytisine, and the competitive antagonist dihydro-β-erythroidine—suppressed the UPR. Interestingly, these ligands had diverse effects on the subunit stoichiometry of the assembly receptors, on the trans-Golgi network, and on the eventual upregulation of plasma membrane nAChRs. These observations provide the first suggestions of a pathway leading from intracellular pharmacological chaperoning to modified gene activation (16).

### Psychiatric Drugs are Candidate Intracellular Ligands

Because they are weak bases, orally available CNS drugs have high membrane permeability, allowing them to pass through the blood-brain barrier, at least in their neutral, deprotonated form. No specific membrane transporter is required. An appreciation for this permeability may be gained by inspecting the record for any drug in PubChem Compound, a sister database to Pubmed. The “logP” or “clogP” entry describes the logarithm of a compound's partition constant between octanol and water. All drugs described in this review have logP values >2, rendering them even more membrane-permeant than nicotine (logP=1.1). Thus, they also readily penetrate into neurons and into organelles. The ER has a pH very similar to that of CSF. The protonation-deprotonation process occurs in microseconds, and both in the extracellular solution and the ER, it is the protonated forms of the drug which usually bind to its receptor.

CNS drugs are designed not to be substrates for various plasma membrane efflux pumps which would remove them from CSF. Thus they remain intracellular and intra-organellar. They bind very tightly to their targets. They are resistant to enzymatic metabolism, thus allowing them to interact with their targets for hours to days. Thermodynamics dominates: within the ER, the drug-receptor interaction spends several minutes, hours, or days finding its lowest free-energy state (which is also the tightest-binding state, explaining how therapeutic effects of psychiatric drugs occur at surprisingly low doses). This tightly bound state is often resistant to ERAD, hiding the hydrophobic domains which would otherwise bind BiP, and thus allowing binding with the vesicle coat protein II complex (COPII) and exit from the ER. The COPII complex comprises five distinct proteins first identified by yeast genetics (10). For this essay, a key protein is Sec24, which binds the transported cargo protein and has four isoforms (a through d) in higher eukaryotes (17).

### Recent Work on GPCRs Relevant to Psychiatric Drugs

Pharmacological chaperoning of psychiatric drug targets would be most important for cases where GPCRs are substantially retained in the early secretory pathway. Early experiments showed that GPCRs have a marked intracellular component (18), for instance in microsomal fractions enriched with markers for endoplasmic reticulum and Golgi (19, 20).

### 5-HT2A and Dopamine D2/D3 Receptors Localize Partially to the ER

The major accepted targets for antipsychotic drugs are dopamine D2/D3 and serotonin 5-HT2A. Systematic and anecdotal observations from many laboratories suggest that D2/D3 receptors remain to some extent in intracellular membranes (21), and probably in the ER (22), even under normal circumstances. Studies of 5-HT2A receptors also show that they are strongly intracellular (23, 24), and are partially localized in ER (25). It cannot yet be claimed that this situation is fully analogous to that for α4β2 nAChRs, but ER and cis-Golgi retention does increase the possibility for ER stress. Dopaminergic neurons may exhibit ER stress even in normal circumstances (26).

Many elegant experiments also show how ligands control the trafficking of 5-HT2A receptors in the late exocytotic/endocytotic pathway (27), consequent on their phosphorylation by receptor kinases and interaction with β-arrestins. However a recent paper suggests that the ability of drugs to induce internalization and down-regulation of 5-HT2A receptors is unrelated to antipsychotic actions (28).

### GPCR Ligands Act as Pharmacological Chaperones

That GPCR ligands, both agonists and antagonists, can act as pharmacological chaperones is an established concept (Supplementary Table 1) (29-40). Previous experiments have concentrated on direct upregulation of the GPCR by pharmacological ligands, with only passing attention (34) to the consequences for ER physiology. Antipsychotic drugs that are GPCR ligands, like other psychiatric drugs, possess pharmacokinetic, binding, and metabolic characteristics that render them highly accessible to the lumen of the ER, and roughly as stable within that lumen as in the extracellular solution. Furthermore the endogenous neurotransmitters are not present within the lumen to compete with the therapeutic ligands. The major classes of GPCR ligands include agonists, antagonists, allosteric modulators, and inverse agonists. As explained above, the ligand-receptor complex can thoroughly explore the energy landscape within intracellular compartments, eventually finding the state of tightest binding. As a result, class A GPCRs display a range of binding states, representing distinct conformations (41, 42). These conformations are stabilized by binding of G proteins, β-arrestin (43), ions, and accessory proteins. Inverse agonism may be the mode in which antipsychotic drugs bind most tightly within the ER.

Evidence for involvement of GPCRs in ER stress is found in a recent C. elegans study, showing that an octopamine receptor, an invertebrate homolog of mammalian adrenergic receptors, is associated with an unfolded protein response (44). The identified genes are part of the “activated in blocked UPR” (abu) pathway, which differs from the classical mammalian UPR pathway. An interesting point of the study is that the consequences of the UPR manipulation are read out in non cell-autonomous fashion, by effects on the animal's immune system.

Several GPCRs participate fully in signal transduction, including G protein activation, when their ligands are added extracellularly and permeate to the ER (45). This full transduction pathway is likely a special case and does not play a major role in the postulated mechanisms of this essay.

### “Matchmaking” in GPCR Homo- and Heterodimers

“Matchmaking” is considerably less developed at GPCRs. That the atypical antipsychotics seem to bind weakly to D2 receptors and more stronger to 5HT2A receptors has been interpreted previously in terms of either cell-autonomous or non cell-autonomous interactions in signaling downstream from inhibition of G protein and/or arrestin signaling. However in the context of possible matchmaking, one should consider whether direct or indirect physical contact occurs between these two GPCR classes, and if so whether such interaction is altered by drugs. Many papers show that class A and class C GPCRs can exist as dimers and in some cases, heterodimerization is required for ER exit (41). Homo- or heterodimerization of GPCRs would be favored while the GPCRs are being concentrated by direct binding to the several dozen Sec24 molecules associated with each COPII vesicle. Of specific interest, heterodimers between D2 receptors and 5-HT2A receptors have been reported (46, 47), but it is not clear whether ligand binding affects the strength of this binding, or indeed any heterodimeric interaction between GPCRs.

Heterodimers between 5-HT2A and a metabotropic glutamate receptor (mGluR), mGluR2, occur in cortical neurons (48, 49). An experimental mGluR2 prodrug, LY2140023, has shown promise for schizophrenia (50-52). In Supplementary Information, we explain how the prodrug strategy (53) yields a ligand that can bind to its target in the early exocytotic pathway. Again, whether the mGluR2 ligand affects the probability of heterodimerization is not known.

Several antipsychotic drugs interact with literally dozens of molecular targets (54). This enhances the possibility for “matchmaking” within the ER, as explained below for SERT.

“Escorting” Effects with GPCRs

“Escorting” is the least developed, but perhaps most powerful, concept for the effects of ER binding to GPCRs. In principle any protein that (a) interacts with GPCRs within the early exocytotic pathway, (b) interacts with other proteins as well, and (c) exists in rate-limiting quantities, might affect cell function if it is differentially “escorted” or “abducted” as a result of ligand binding to GPCRs. The GPCR-associated sorting proteins (GASP) family should be studied in this regard (55). Homer proteins, when overexpressed may interact with mGluRs in the ER (56). Receptor-associated membrane proteins (RAMPs) (57) and major histocompatibility proteins (58) present other possible families. If the escorted protein becomes secreted, non cell-autonomous results are possible.

### Side Effects of GPCR Drugs

That psychiatric drugs activate off-target receptors has long been suspected as the cause for agranulocytosis, weight gain, and other side effects. These side effects could also arise from intracellular binding.

That both agonists and antagonists of D2 receptors lead to several types of dyskinesias is usually ascribed to circuit-based phenomena. We point out that both agonists and antagonists could act as pharmacological chaperones of GPCRs. A partially analogous situation, in which both agonists and antagonists of nAChRs act as pharmacological chaperones to suppress ER stress, has been described at nAChRs (16). Thus pharmacological chaperoning, matchmaking, escorting, abduction, or UPR-related changes in gene activation could cause some dyskinesias.

### Recent Work on SERT

Raphe neurons are the major neuronal type that express SERT, the primary target of SSRIs. In Raphe neurons, most axonal SERT is on the PM; but most SERT is cytoplasmic in the soma and dendrites of neurons, in platelets, and in astrocytes (59-61). Thus, intracellular events such as chaperoning, matchmaking, and escorting have the potential to alter SERT biology.

Chaperoning is an established concept at SERT. The “alternating access” model of neurotransmitter transport (62) summarizes the concept that neurotransmitter transporters shuttle between two major conformations, in which a central region of the transporter faces either the cytosolic or cytoplasmic/ER lumenal compartment. Within and between each of these two orientations, several substates occur. For neurotransmitter transporters as for other proteins, “by saturating SERT with a ligand, we shift the equilibrium toward protein conformations optimal for binding that ligand” (63). The most stable state often has the greatest resistance to degradation. Each class of ligands favors distinct conformations, in some cases specifically favoring posttranslational modification, trafficking, and interaction of the transporter with regulatory proteins (63). These conformations are stabilized by binding of substrates such as Na⁺, Cl⁻ and K⁺, which themselves, have different concentrations in the cytoplasm vs ER lumen vs extracellular space. Binding of fluoxetine has been best studied, and in its presence the most stable conformation appears to be midway between the extracellular/lumenal-facing and cytosol-facing orientation of the permeation pathway (63). There is evidence that even among the SSRIs, the various ligands interact differentially with the binding of the inorganic co-substrates (for instance fluoxetine vs paroxetine) (64). These differences add to the possibilities for explaining how subsets of patients are benefited by distinct SSRIs.

Within the ER, neurotransmitter transporters interact with the protein chaperones calnexin, calreticulin, and BiP. This binding facilitates both intra- and intermolecular arrangements of hydrophobic segments (65, 66). Presumably like most molecules that exit the ER, the transporter must be correctly folded before binding to Sec24c or other members of the COPII cargo-binding protein Sec24 family (67). As noted, SSRIs may favor this specific form of chaperoning. Ibogaine, a hallucinogenic alkaloid, has complex pharmacology including blockade of SERT and the dopamine transporter. At SERT, ibogaine acts as a pharmacological chaperone to rescue some ER exit mutations (67). Interestingly, SERT appears to be the only neurotransmitter transporter studied to date that specifically requires Sec24c to exit the ER. The structural basis for this difference is not known (68), but that neuronal proteins specifically utilize individual Sec24 isoforms has precedents in the ER exit literature (69, 70). This concept also applies to neuronal proteins (68, 71).

“Matchmaking” is less well established at SERT. SERT and neurotransmitter transporters dimerize in the ER as a prerequisite for binding to Sec24 (68). Ligands for SERT enhance ER exit, but it is not known whether SSRIs affect this dimerization. Once dimerized, mutations in the C-terminus of SERT impair its ability to bind to Sec24 proteins (67).

What are the “escort” consequences of enhanced ER exit of SERT? Baudry et al (72) showed that an SSRI suppresses microRNA16 and enhances S100β exit. Other research suggests a role for p11 (S100A10) (73). Most researchers postulate that these are consequences of excess extracellular serotonin (74). Within the framework of the present review, one would suggest that these actions arise via suppression of ER stress and/or escorting or abduction.

### Axonal Transport

The effects of psychiatric drugs have long been assumed to involve gene activation; and the ER stress/UPR pathway has direct influences on gene activation; but this process takes just a day or two. The “inside-out” view, in which therapeutic effects result from action in the ER, leads one to emphasize the role of intracellular trafficking during the several weeks involved in the full action of antipsychotic and antidepressant drugs. Some effects occur within days, perhaps because of effects that occur in the soma; on an intermediate time scale, events might involve transport to dendrites. Axonal transport occurs at a rate of ˜1 mm/day, accounting for the most delayed effects, for instance ˜30 days at the axon terminal of a 3 cm human axon. This might be briefer in animals with shorter axons. No retrograde transport need be invoked.

### “Rapid” Antidepressant Effects of Ketamine Occur in Dendrites

There is a previously suggested mechanistic framework (75-77) for the antidepressant effects of ketamine, which become established within 1-2 hr. Mechanisms within the framework of this review begin with the binding of ketamine to nascent NMDA receptors in the dendritic ER.

The intracellular binding explanation for the 1-2 hr effects of ketamine seems at first glance inconsistent with the idea that intracellular binding is also the source of the 2-3 week time course of SSRI antidepressant action. It is therefore a key observation that these antidepressant effects vanish in Val/Val and Met/Met BDNF knock-in mice, which cannot transport BDNF message to dendrites (77). One straightforward interpretation: whereas other psychiatric drugs require protein synthesis at the somatic compartment, followed by transport to axons, the ketamine actions are local to dendrites, so that the newly synthesized or “escorted” BDNF can be secreted near dendrites within minutes. Dendrites have ribosomes, ER, endoplasmic reticulum exit sites, and Golgi, and are fully competent to translate and secrete BDNF, without requiring the soma (78-80). Supplementary Information discusses local ketamine concentrations (76, 81-85).

### Testing and Applying Inside-Out Hypotheses

We must test hypotheses that intracellular binding in the early exocytotic pathway explains actions of antipsychotic and antidepressant drugs. The compartmentalization of drug actions must be studied, presumably with isolated neurons. The extent to which drugs bind within the ER could be tested most generally with stable isotope-labeled drugs and nanometer scale secondary ion mass spectrometry. The compartment in which drugs act could be studied by quaternizing the amines to reduce membrane permeation (86). Compartmented cultures also seem well suited for such research (87).

Kinetic experiments often shed light on mechanism. One could visualize intracellular movements of receptors, transporters, and “escorted” or “abducted” proteins as they travel from the ER to axons and dendrites in response to drug actions (16, 88). Time-resolved proteomic and transcriptomic studies could test whether antipsychotic and antidepressant drugs produce early effects on ER stress and UPR pathways. These would be conducted both in the soma, for GPCRs and SSRIs, and in dendrites for ketamine.

Cell-free systems can report on drug-receptor interactions. Few contemporary papers continue to report the binding of psychiatric drugs to purified endoplasmic reticulum from brain (19, 20); but this seems crucial for understanding how each class of agonist, antagonist, inverse agonist, open-channel blocker, or allosteric modulator produces chaperoning and/or matchmaking at GPCRs, transporters, or ligand-gated channels. In analogous experiments at nAChRs, agonists chaperone and upregulate at concentrations far lower than required to activate, but far higher than the equilibrium binding constant (16). For antagonists as well, the chaperoning effects occur at concentrations far higher than the equilibrium binding constant. Evidently the chaperoned state(s) require further characterization. Because we suggest that key chaperoning events occur at the stage of ER exit and ERGIC, experiments should incorporate reconstituted systems for membrane budding and fusion (10).

If the hypotheses gain further support, psychiatric diseases will belatedly join the list of diseases that are approached therapeutically by manipulating early exocytotic pathways (2, 89). Strategies for designing more effective psychiatric drugs could incorporate the experiments described in this section. An appropriate challenge would be to decrease the cognitive deficits of schizophrenia (6), perhaps by enhancing the early exocytotic pathways of neocortical chandelier and basket cell somata. Although these cells have relatively short axons, their complex and numerous axonal terminals might place heavy demands on early exocytotic pathways (90, 91).

### Comment on Pathophysiology

Three large classes of proteins may participate in pharmacological chaperoning, matchmaking, escorting, “abduction”, ER stress, unfolded protein responses, and related mechanisms. (a) One-third of a typical cell's protein species enter the ER. (b) Many additional cytoplasmic protein species govern proteostasis of ER proteins and transport of vesicles in the early and late endocytotic pathways. (c) Many additional proteins enter the nucleus to govern chromatin structure, initiation and transcription of the genes, and mRNA processing of the proteins in class (a) and (b). A sizeable fraction of human genes encode neuronal proteins in class (a), (b), or (c), and non-coding regions may also play a role. Malfunctions in classes (a), (b), or (c) could well lead to a deterioration of neural function.

A marked, but neither apoptotic nor necrotic, deficit of important proteins that pass through the ER could account for the observations that reduced gray matter volume occurs in schizophrenia, depression, or bipolar disease, but also that this reduction is more subtle than in neurodegenerative disease. The accompanying functional deterioration might also be subtle enough to avoid ER stress and/or UPR activation, but resilient function (92) could still be partially restored by the pharmacological chaperoning, matchmaking, or abduction effects of existing antipsychotic and antidepressant drugs. Even if each hypothesis of this paper is proven, we would hesitate to infer that all psychiatric patients have a neuronal population showing “cellular stress”, according to this phrase's contemporary biomedical connotations. Therefore, inside-out therapeutic mechanisms are compatible with the idea that various polygenic, multifactorial, and partially penetrant processes underlie psychiatric diseases. However, this compatibility remains vexingly general.

References Cited in Example 2:

The references cited in Example 2 are listed in Lester, et al., Biol Psychiatry. 2012 Dec. 1; 72(11):907-15, the entire contents of which are incorporated herein by reference.

### Example 3

### Chronic Pre-Treatment of Dopaminergic Neurons with a Smoking-Relevant Concentration of Nicotine Prevents Nuclear Upregulation of ER Stress Markers

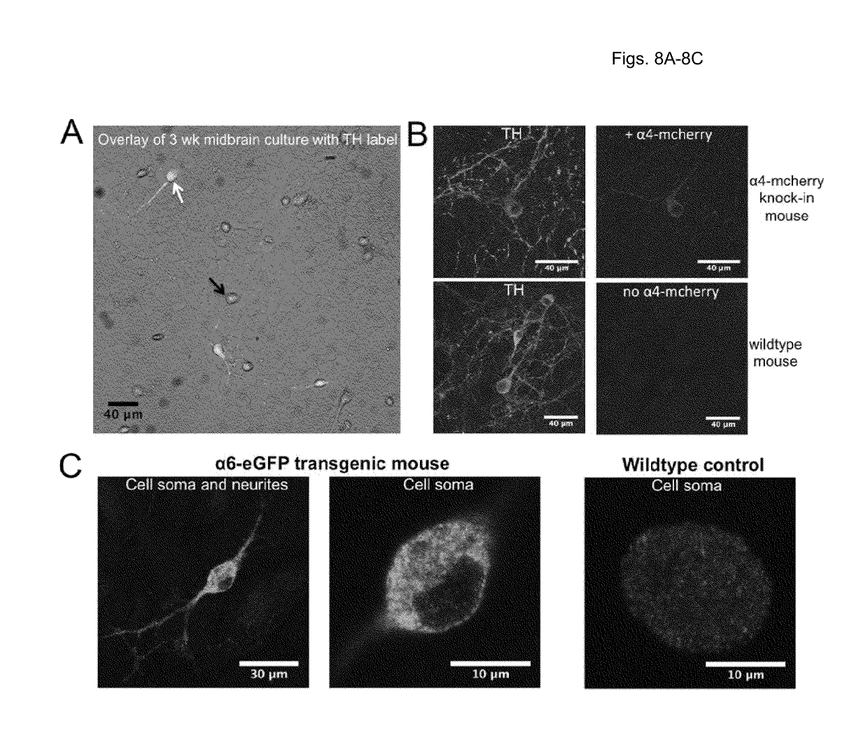

This Example demonstrates that cultured mouse dopaminergic neurons expressing endogenous neuronal nicotinic acetylcholine receptors (nAChRs) exposed to chronic pre-treatment with nicotine are protected from an induced ER stress response. We routinely culture mouse dopaminergic neurons and have demonstrated that these neurons contain native α4* and α6* nAChRs within intracellular compartments (* denotes other uncharacterized subunits present in the receptor pentamer). Three-week-old mouse ventral midbrain cultures were obtained from 14 day old mouse embryos. The ventral midbrain neurons, indicated by arrows in FIG. 8A, lie on a monolayer of glial cells. The culture was immunostained for tyrosine hydroxylase (TH) to identify dopaminergic neurons. The white arrow points to a TH positive dopaminergic neuron and the black arrow points to a TH negative GABAergic neuron. The average yield of dopaminergic neurons in these cultures is ˜30%. Representative images of cultured dopaminergic neurons obtained from a transgenic mouse with a mcherry fluorescent protein-tagged α4 nAChR subunit are shown in FIG. 8B. TH positive neurons show that these neurons express α4* nAChRs. TH positive neurons from parallel cultures of wildtype mouse embryos do not show mcherry fluorescence. FIG. 8C shows a representative image of a midbrain dopaminergic neuron obtained from a transgenic mouse with an eGFP fluorescent protein-tagged α6 nAChR subunit, immunostained for eGFP. α6* nAChRs are present intracellularly, within the cell soma and the neurites. Control cultures obtained from wildtype mice do not show eGFP fluorescence. The haze in wildtype neurons is non-specific staining by the secondary antibody.

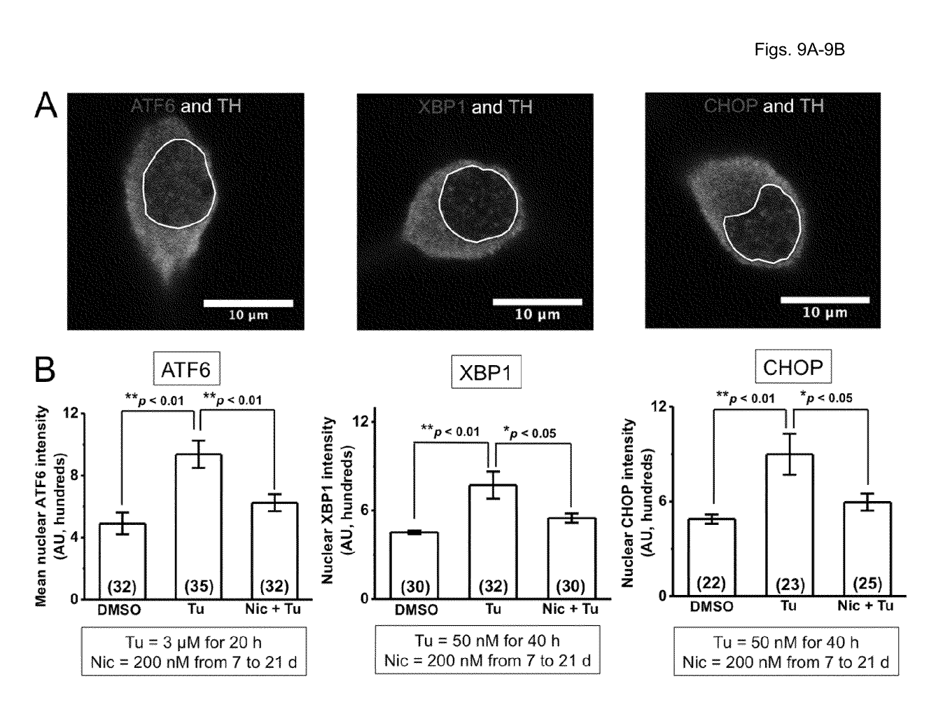

FIG. 9 illustrates that chronic exposure to nicotine prevents the upregulation of ER stress markers, ATF6, XBP1 and CHOP in cultured dopaminergic neurons treated with Tunicamycin, a known inducer of the ER stress response. In a series of experiments, 3 week-old wildtype cultured mouse ventral midbrain neurons were either untreated or exposed to 200 nM nicotine (Nic) from days 7 to 21. Separate dishes were then treated with Tunicamycin (Tu) or DMSO (vehicle) and immunostained for TH and either ATF6 or XBP1 or CHOP. ER stress was assessed by quantifying the average intensity of the ER stress marker (ATF6/XBP1/CHOP) within the nucleus of TH labeled dopaminergic neurons. The absence of TH label in the nucleus of these neurons was used as a guide to manually demarcate the nucleus and average nuclear intensity was quantified using ImageJ software. FIG. 9A shows representative confocal images of dopaminergic neurons showing TH staining (primarily cytoplasmic) and the indicated ER stress marker (primarily nuclear). The white line shows manually demarcated nuclei for each neuron. Graphs of average nuclear intensity from neurons in DMSO, tunicamycin (Tu) or nicotine (Nic) and tunicamycin (Tu) treated culture dishes are shown in FIG. 9B. The ER stress marker that is quantified is indicated at the top of each graph and the concentration and time points for nicotine and tunicamycin treatments are indicated at the bottom of each graph. Numbers of cells imaged are indicated in parentheses for each column and p values are based on a 2-tailed t-test; error bars are ±S.E.M.

These data clearly show that chronic 2 week pre-treatment of dopaminergic neurons with a smoking-relevant concentration of 200 nM nicotine prevents the nuclear upregulation of ER stress markers, ATF6, XBP1 and a pro-apoptotic marker, CHOP, in dopaminergic neurons expressing intracellular nAChRs.

### Example 4

### Trafficking of α4* Nicotinic Receptors Revealed by Superecliptic Phluorin: Effects of a β4 ALS-Associated Mutation and Chronic Exposure to Nicotine

This Example employs a pH sensitive GFP analog, superecliptic phluorin, to observe aspects of nicotinic acetylcholine receptor (nAChR) trafficking to the plasma membrane (PM) in cultured mouse cortical neurons. The experiments exploit differences in the pH among endoplasmic reticulum (ER), trafficking vesicles, and the extracellular solution. This Example also demonstrates a method to quantify ER exit sites in neurons. This method differs from methods described in other Examples herein, in that for neurons, ER exit sites are best quantified from the processes (dendrites) rather than the soma because the large number of ER exit sites on the neuronal soma precludes accurate quantification.

The data confirm that few α4β4 nAChRs, but many α4β2 nAChRs, remain trapped in neutral intracellular compartments, mostly the ER. We observed fusion events between nAChR-containing vesicles and PM; these could be quantified in the dendritic processes. A β4R348C polymorphism has been linked to amyotrophic lateral sclerosis (ALS). This mutation depressed fusion rates of α4β4 receptor-containing vesicles with the PM by ˜2 two-fold, with only a small decrease in the number of nAChRs per vesicle. The mutation also decreased the number of ER exit sites, showing that the reduced receptor insertion results from a change at an early stage in trafficking. We confirm the previous report that the mutation leads to reduced agonist-induced currents; in the cortical neurons studied, the reduction amounts to 2-3 fold. Therefore the reduced agonist-induced currents are caused by the reduced number of α4β4-containing vesicles reaching the membrane. Chronic nicotine exposure (0.2 μM) did not alter the PM insertion frequency or trafficking behavior of α4β4-laden vesicles. On the other hand, chronic nicotine substantially increased the number of α4β2-containing vesicle fusions at the PM, representing a final stage in α4β2 nAChR upregulation. Superecliptic phluorin provides a tool to monitor trafficking dynamics of nAChRs in disease and addiction.

### Methods & Results

Details of the methods employed and the Figures referenced in Example 4 are provided in Richards, et al., 2011, J. Biol. Chem. 286(37):31241-31249, the entire contents of which are incorporated herein by reference. Numbers in parentheses denote references listed in Richards et al., 2011.

We used superecliptic phluorin (SEP) fused to the C-terminus of alpha4 nicotinic receptors (nAChRs) to examine the subcellular distribution and trafficking dynamics of receptors expressed in cortical neurons. SEP is a pH-sensitive version of green fluorescent protein (GFP) that undergoes 488 nm excitation at neutral pH (˜7.4), but loses this excitation maximum under acidic conditions (12). As a result, SEP neither photobleaches nor fluoresces at low pH but regains its fluorescence at neutral and basic pH. Tagging of α4 on the extracellular C-terminus results in the orientation of SEP on the lumenal side of most organelle types in the secretory pathway. While the endoplasmic reticulum (ER) and cis-Golgi have lumenal pH of ˜7.2 and ˜6.7, respectively, the lumen of both the trans-Golgi network and secretory vesicles is much more acidic (pH<6.5) (39). If one illuminates via total internal reflection (TIRF) microscopy, only those receptors resident in nearly neutral organelles close to the plasma membrane, or inserted in the plasma membrane, are visible within the excitation field. Thus, receptors in the later stages of the secretory pathway neither fluoresce nor photobleach due to the acidic environment. This arrangement allows us to visualize receptors as they are inserted into the plasma membrane, because the SEP moiety transitions from the acidic environment within the transport vesicle to the neutral pH of the extracellular solution.

To verify that α4-SEP subunits form functional receptors we compared whole cell currents of α4-SEP β2-wt and α4-eGFP β2-wt in transiently transfected Neuro-2a cells (FIGS. 1A and B of Richards et al.). The density of ACh-induced currents for α4-SEP β2 nAChR were 40% that seen for receptors with the M3-M4 insertion of eGFP (FIG. 1B of Richards et al.). These data show that the receptors with a C-terminus FP fusion function, although less well than the control. These cells had similar membrane capacitance (˜20 pS).

An epifluorescence image of a representative cortical neuron transfected with α4-SEP β4wt reveals fluorescence from all non-acidic compartments, i.e., from receptors at the membrane, in the ER, and in the cis-Golgi (FIG. 2A of Richards et al.) (Untransfected cells showed no detectable fluorescence). In order to determine the extent of PM expression, we adjusted the extracellular pH to 5.4. Because SEP is fused to the C-terminus of α4, it senses the extracellular solution; therefore a reduction in the pH results in the elimination of the membrane based fluorescence signal. Emission from receptors residing in intracellular regions is initially unaffected by the change in extracellular pH. On addition of an acidic extracellular solution the fluorescence intensity in the soma is clearly reduced (FIG. 2B of Richards et al.) but is still quite prominent, indicating that a major population of receptors resides in the ER. This observation is entirely consistent with previous data for α4β2 nAChRs in neurons and does not arise from artifacts due to tissue culture, trafficking, or microscopy (10, 40, 41). The distribution of receptors in the processes is rather different: many dendrites visible in the original image exhibited distinctly lower levels of fluorescence with the pH reduction. While receptors are still clearly visible in the processes, this marked fractional decrease in the fluorescence indicates that the processes have a proportionally smaller population of α4β4 nAChRs in neutral environments such as ER compared to the cell body. Because these images are taken with epifluorescence, even receptors residing 1 to 2 μm away from the PM would still be visible.

FIGS. 2C and D of Richards et al. show TIRF images of the same neuron. The major observation is that the neuron exhibits robust fluorescence, with a typical membrane “footprint” pattern, at the neutral solution; but that this fluorescence is markedly diminished with the acidic solution. To verify that intracellular fluorescence originates from receptors retained in the ER, we cotransfected neurons with nAChRs and a dsRed labeled ER localized sequence (KDEL). Comparison of the nAChR signal (GFP) and the ER signal reveal extensive colocalization (Supplemental figure S1 of Richards et al.). This indicates as expected that the intracellular fluorescence can be contributed to ER resident receptors. The data thus confirm and extend those of an earlier study, using fluorescent nAChRs with an alternative ER marker, which concluded that most intracellular nAChRs reside in the ER (8). It is possible some ER fluorescence also arises from unassembled or partially assembled subunits; but SEP does not fluoresce in those subunits that are transported to lysosomes and other acidic catabolic vesicles.

We comment on the comparison between the epifluorescence (FIG. 2A of Richards et al.) and TIRF (FIG. 2C of Richards et al.) images. (1) The two images emphasize different aspects of cell structure. Where the TIRF image is relatively brighter than the epifluorescence image, we assume that most of the nAChRs reside near the coverslip. Some portions of dendrites are visible in epifluorescence but not in TIRF; presumably these regions extend away from the surface of the coverglass. (2) Typically the TIRF excitation field extends ˜200 nm in the axial direction. As a result only the ER at the periphery of the cell is visible along with the membrane fluorescence. With an acidic extracellular solution, TIRF excitation reveals that most of the fluorescence has been quenched, leaving a small but measureable fraction of residual fluorescence in the soma. This reduction in fluorescence in TIRF images at acidic pH suggests that the majority of intracellular receptors are not in near-membrane neutral organelles but reside much deeper, and that any dendritic ER contains low levels of α4β4 nAChRs.

If changes to extracellular pH do eventually affect intra-organellar pH, this distortion would presumably occur most strongly for organelles near the PM. This is a particular concern in the processes, as the small radius dimensions places most of the organelles <1 μm from the extracellular solution. To test the effects of the acidic extracellular solution on SEP fluorophores in the ER of the soma and dendrites, we performed a series of control experiments. As unassembled α4 subunits in the absence of a complementary beta subunit are trapped in the ER, we transfected neurons with only the α4-SEP. We then compared the fluorescence at neutral and acidic pH (Supplemental figure S2 of Richards et al.). These controls showed no observable decrease in the fluorescent signal in either the soma or the dendrites. Therefore the acidic extracellular solution on the time frame of our experiments does not affect the pH of the ER. These points establish that it is feasible to employ SEP-nAChR fusions to measure surface insertion of nAChRs.

ER Exit and Plasma Membrane Insertion of α4β4 nAChRs are Reduced by the Rβ348C Mutation

We introduced an Arg to Cys point mutation in the mouse β4 construct at position 348, a position adjacent to a putative LFM ER exit motif in the M3-M4 cytoplasmic loop. The aligning mutation in the human β4 is associated with ALS and yielded reduced ACh- and nicotine-induced currents when compared to the wild type construct (28). While a previous study shows that the mutation results in reduced electrophysiological activity likely through reduced expression, it is not clear if this results from decreased trafficking of receptors to the PM. To determine if the observed reduction of currents seen with the β4 mutation was due to an altered number of α4β4 nAChRs (presumably a decrease), we transiently transfected cortical neurons with α4-SEP β4-wt and compared them to neurons transfected with an equal amount α4-SEP β4R348C. To gain further insight into differences in the subcellular distribution of α4β4, we took advantage of the pH sensitivity of the superecliptic phluorin tag on the C-terminus of the α4 construct.

We were able to distinguish differences in the subcellular distribution of wt and mutant α4β4 receptors utilizing the pH sensitivity of the SEP moiety on the C-terminus of the α4 construct. While changes in total levels of fluorescence are more subtle than the electrophysiological studies that showed reduced agonist-induced currents for β4R348C receptors (28, 29), surveying individual cells at both neutral and acidic pH eliminates intensity based bias originating from large variations in expression levels among cells. Here we also used ROIs to discriminate between the cell body and processes. Calculating the ratio of fluorescence at pH 5.4 to pH 7.4 provides a measure of the extent of receptors in the ER as compared to those in the plasma membrane, and is unaffected by receptors that are being actively trafficked to the plasma membrane as they are not fluorescent. We found that ˜75% of the observable fluorescence in the soma of both wildtype and mutant receptors was from plasma membrane resident receptors. Additionally, we found that the majority of the fluorescence in the dendrites was quenched at low pH to the point the processes became indistinguishable from the background. This suggests that there were no differences in the subcellular distribution of the mutant and wildtype receptors and in both cases receptors reside at the plasma membrane and are not retained in the dendritic ER and/or cis-Golgi.

As superecliptic phluorin is not excited in an acidic environment, it does not undergo photobleaching during vesicular transport, but exhibits a burst of fluorescence when exposed to the neutral extracellular solution as the vesicle merges with the PM during receptor insertion. This can be exploited to reveal trafficking dynamics. Additionally, the relative brightness of each insertion shows whether observed differences in plasma membrane levels result from an increase in the number of vesicles or an increase in the number of receptors per vesicle. To compare trafficking rates of α4β4wt and α4β4R348C we used TIRF microscopy to monitor insertion rates in the processes of cortical neurons.

As seen from the pH dependence studies (FIG. 2 of Richards et al.) only a small fraction of the fluorescence observed in dendritic processes is due to ER and cis-Golgi resident nAChRs. In TIRF microscopy insertion events are more readily visualized in the presence of lower levels of background fluorescence, and as a result insertion events in the processes are detected without interference from intracellular emission. We compared trafficking dynamics for the wild type and R348C mutant nAChRs.

FIG. 3A of Richards et al. shows a dendrite of a cortical neuron transfected with α4-SEP β4-wt and imaged with TIRF microscopy. A clear insertion event occurs at the plasma membrane evidenced by the bright punctate feature. Three consecutive frames with 200 ms exposure reveal the nature of the insertion event. The initial frame (FIG. 3B of Richards et al.) shows no bright features while the next frame (200 ms later) (FIG. 3C of Richards et al.) shows the arrival of a vesicle at the PM, which is apparent from the bright fluorescent feature outlined by the white circle. Just 200 ms after its arrival the vesicle has now recycled from the membrane as evidenced by the absence of the fluorescence feature. The transient nature of the arrival event and the lack of observed spreading of the fluorescence from the concentrated punctate region indicate that the vesicle briefly encounters the PM and does not transfer all its nAChRs to the PM.

We quantified these data by counting the number of insertion events in the dendrites of the cortical neurons per linear distance of the dendrite per time increment. These data reveal clear differences between the wt receptors and the β4-R348C* mutant receptors. FIG. 3E of Richards et al. shows that the wt receptors are inserted at nearly twice the rate of the mutant channels. From this it is clear that the lower agonist-induced currents observed for cells transfected with the mutant receptors (28) arises at least partially from differences in trafficking to the PM.

The intensity of the fluorescence burst associated with the insertion is proportional to nAChR numbers on the vesicles. We studied this parameter by taking the average fluorescence of the area demarcated by the ROI of the insertion from the slide preceding the arrival event and then subtracting this from the frame with the insertion event. FIG. 3F compares the intensity of the insertion events for the wild type to the mutant receptors. The insertion events for the wild type receptors show slightly higher intensity than the mutant receptors. It should be noted that >90% of events are transient interactions whose fluorescence pulse lasts <1 s; despite the larger fluorescence intensity there is no clear method for precisely determining the contribution of fluorescence from inserted receptors and how much is recycled away from the plasma membrane.

To examine if the longer persistent events (several s) resulted in the insertion of all receptors, we examined the time course of each event. FIG. 4 of Richards et al. shows such a persistent insertion event. The consecutive frames taken every 200 ms show a clear insertion event that lasts for more than one s, and the punctate increase becomes more diffuse as it spreads into the plasma membrane (FIG. 4H of Richards et al.). While persistent events represented only a few percent of the total number of insertions, the majority of the longer interactions exhibited a diffusion of receptors away from the original puncta associated with the insertion event. This indicates that the more prolonged fusion events could, indeed, account for most of the receptors deposited on the membrane.

As LFM motifs have been implicated in ER export of cargo, it is possible that a mutation in such close proximity to an LXM motif disrupts the normal trafficking through the secretory pathway. To further investigate this idea, we examined the effect of the β4-R348C mutation on ER exit sites (ERES), which have been shown to increase and decrease with mutations in ER exit and retention motifs of α4β2 receptors (8). To observe differences in ERES, cortical neurons were co-transfected with α4-GFP β4-wt and the ERES marker, Sec24D labeled with mCherry. Confocal microscopy was used to observe areas where Sec24D has been recruited into ERES in the processes of neurons as seen in FIG. 5 of Richards et al. ERES were defined as punctate regions which exhibited brighter fluorescence than the background mCherry fluorescence in the rest of the dendrite. FIG. 5 of Richards et al. depicts a neuron transfected with α4-eGFPβ4-wt and Sec24D-mCherry. Several ERES can readily be observed throughout a major portion of the dendritic processes. The white line in FIG. 3A of Richards et al. indicates the ROI used to distinguish the cell soma from the processes and only ERES outside of the cell body were counted. Compared to mutant α4β4 nAChRs, we found significantly more ERES for cells expressing the wt nAChRs. These differences further suggest that the ER exit of assembled α4β4 pentamers is interrupted by the R348C mutation.

### Agonist Evoked Currents are Reduced by the Rβ348C Mutation

That the wt receptors are inserted at nearly twice the rate of the mutant channels (FIG. 3E of Richards et al.) does not explain the ˜10-fold difference in agonist-induced currents previously reported when the wt and mutant nAChRs were compared by expression in a clonal cell line (22, 23). To correlate the fluorescence based insertion measurements with electrophysiological measurements, we performed whole-cell patch clamp recording from neurons in the preparation used for our fluorescence experiments, transfected either with α4-GFPβ4-WT or with α4-GFPβ4-R348C nAChRs. The successfully transfected neurons were identified with green fluorescence (FIG. 6. A, D of Richards et al.). Puffs of 300 μM ACh (0.1 s, 20 psi), typically give maximal currents in previous experiments and therefore provide a measure of nAChR number. ACh evoked inward currents in 100% (8/8) and 92% (11/12) of neurons transfected with α4-GFPβ4-WT and α4-GFPβ4-R348C nAChRs, respectively (FIG. 6B, 6E of Richards et al.). The α4-GFPβ4-R348C nAChR currents (120±33 pA, n=12) were only 40% of α4-GFPβ4-WT nAChR currents (310±72 pA, n=8) (p=0.02) (FIG. 6C of Richards et al.). Because variations among the size of neurons could complicate the data, we calculated current density (dividing ACh currents divided by membrane capacitance). The current density of α4-GFPβ4-WT nAChRs (6.3±1.4 pA/pF, n=12) was 38% of that of α4-GFPβ4-R348C nAChRs (16.3±2.1 pA/pF, n=8) (p=0.001) (FIG. 6F of Richards et al.). Thus in cortical neurons, the R348C mutation produces a fairly consistent ˜2-fold decrease in α4β4-insertion rate and ˜2.5 fold decrease in ACh-induced currents.

Chronic Exposure to Sub-μM Nicotine Increases Insertion of α4β2 nAChRs

Nicotine acts as pharmacological chaperone for the assembly of some subtypes of nicotinic receptors such as α4β2. Previous electrophysiological, biochemical, and fluorescence based measurements demonstrate that the exposure to physiologically relevant nicotine concentrations increases the numbers of receptors on the PM (8, 35). On the other hand less is known about the effects of nicotine on α4β4. To determine if nicotine has a similar affect on the rate of trafficking of α4β4 receptors, we performed TIRF-based vesicle insertion measurements using α4-SEPβ4 nAChRs. In FIG. 7 of Richards et al. counting the number of insertion events as described previously shows that nicotine has no effect on the insertion rate of α4β4 nAChR containing vesicles. This suggests that nicotine likely does not act as a chaperone helping the assembly of this nAChR subtype.

To extend previous results on nicotine-mediated α4β2 upregulation, we also performed PM insertion studies on α4-SEPβ2 nAChRs. First, we found that a greater fraction of α4-SEPβ2 nAChRs reside in neutral compartments in dendritic processes (fluorescence at pH 5.4/fluorescence at pH 7.4=0.61), presumably because α4β2 nAChRs exit the ER rather inefficiently (8). This shows that a significantly higher portion of the α4β2 receptors are in the ER, compared to α4β4 receptors (Supplementary figure S3 of Richards et al.). Observation of insertion events in α4β2 are complicated by this much larger population of ER resident receptors that exist both in the soma and the processes as compared to α4β4. Nonetheless, we found an additional contrast to the data for α4β4 nAChRs. Experiments performed on α4β2 show that chronic nicotine (200 nM for 24 hr) results in almost a two fold increase in observed insertion events at the membrane (FIG. 7B of Richards et al.). Similar to the insertion events observed for α4β4, those in cortical neurons with α4β2 are primarily transient lasting for less than a second. It has been suggested that nicotine preferentially assembles α4β2 receptors with a (α4)2(β2)3 stoichiometry and further, chaperones these receptors through the secretory pathway, resulting in receptor upregulation at the PM (8). While these experiments show that the well established upregulation of α4β2 does indeed involve an increased number of insertion events in the dendritic processes, they are uninformative with respect to redistribution of receptor stoichiometries.

Additional experiments examined the effect of acute nicotine at a higher concentration (1 μM) on α4β4 receptors as well. While these receptors were not upregulated we did observe a reduction of the number of receptors in the membrane as observed by a loss of fluorescence from the SEP fluorophore giving an Fnic/F0 of 65%+/−0.03 (Supplemental FIG. 4 of Richards et al.).

### Discussion

The fusion of superecliptic phluorin to the C-terminus of the α4 nAChR subunit provides the means to observe receptor delivery to and retrieval from the PM. These labeled α4 subunits do produce nAChRs with modest functional deficit (FIG. 1 of Richards et al.); but their general behavior is consistent with that of the previously studied, fully functional subunits containing fluorescent protein inserted in the intracellular M3-M4 loop (8-10, 42, 43).

The pH sensitivity of SEP allowed us to directly visualize nAChR-containing vesicles arriving at the PM (FIGS. 3 & 4 of Richards et al.) and also to examine subcellular distributions by selectively eliminating the emission from receptors residing at the PM (FIG. 2 of Richards et al.). We utilized these new constructs to examine differences in trafficking as affected by two conditions: R348C, a mutant that has been associated with sALS; and incubation in low concentrations of nicotine.

### Wild Type β2 and β4 Subunits Produce Distinct Distributions in Neurons

Comparing images of α4β4 at both pH 5.4 and 7.4 shows that a majority of the fluorescence visible in TIRF is generated from PM resident receptors rather than those in the ER (FIG. 2 of Richards et al.). The dendrites and soma differ in their patterns of nAChR localization. Acidification of the extracellular solution almost completely eliminated fluorescence in dendrites, indicating that the vast majority of receptors reside on the membrane (FIG. 2 of Richards et al.). On the other hand, a large population of α4β2 is still maintained in dendritic ER.

### An ALS-Associated β4 Subunit Mutation Affects α4β4 Trafficking

The R348C mutation yields reduced agonist-induced currents as compared to wild type receptor, suggesting that either trafficking to the PM is hindered or the gating of individual receptors is compromised (28). Although in all studies cortical neurons transfected with α4-SEP β4-wt exhibited higher average fluorescence intensities in both the cell body and processes as compared to those transfected with α4-SEP β4R348C, these results were too variable for systematic study.

Recently, targeted mutations in the β2 subunit led to increased trafficking out of the ER and greater expression at the PM (8). In part these studies mutated the β2 subunit to mimic the LXM motif found in β4, showing that this motif is a key part of the sequence interacting with cellular machinery to permit trafficking through the secretory pathway. The β4R348C mutation is one amino acid away from this LXM motif. A disruption in the amino acid sequence close to an important export motif could potentially disrupt its normal function.

Trafficking out of the ER also plays a major role in nicotine induced upregulation of α4β2 receptors, as assessed by changes in the number of ERES (8). We now find a reduced number of ERES for the mutant β4 as compared to the wt subunit. Formation of ERES is in part dictated by the amount of cargo requiring transport, which suggests here that the mutant β4 interrupts trafficking early in the secretory pathway likely just after receptors are assembled in the ER. This conclusion is bolstered by additional experiments that showed no modification of the size or brightness of the trans-Golgi network, labeled with Galactosyltransferase-mCherry when we compared cortical neurons transfected with wt and mutant β4.

To determine if the mutation in the M3-M4 loop of β4 led to differences in trafficking dynamics, we also examined the frequency with which nAChR transport vesicles arrived at the PM. Monitoring the rate of insertion for both α4-SEPβ4-wt and α4-SEPβ4R348C revealed differential PM insertion dynamics. wt receptors exhibited almost twice the insertion rate of the mutant receptors in dendritic processes (FIG. 3 of Richards et al.). Additionally, measurements of the average intensity of insertion events also revealed that vesicles carrying wt nAChRs had modestly more receptors than those carrying the mutant. Together, these data show that the increase in ERES correlates with improved trafficking of receptors to the PM. Agonist evoked currents of the mutant receptor were ˜2.5 fold smaller than for the wild type receptor (FIG. 6 of Richards et al.), compatible with our fluorescence measurements. The similarly reduced electrophysiological response and insertion rates suggest that the mutation alters trafficking and not gating of these receptors. The 2-3 fold differences observed here do not match the 10 fold loss of currents seen in previous studies in a clonal cell line (28). Evidently the effect of the mutations depends on the repertoire of associated molecules present in the host cell; perhaps the present neuronal system has more relevance.

While the observed vesicle trafficking events mediate the insertion of nAChRs into the PM, other factors such as the duration of this interaction also influence the number of receptors inserted into the PM. One possibility is that transient interaction of the trafficking vesicle with the PM allows only a fraction of the receptors to insert per vesicle fusion. Like those seen in FIG. 3 of Richards et al., the majority of insertion events are transient. The observed event interacted with the PM for <0.5 s before recycling away from the PM. The relative intensity of the insertion events for the wt construct is larger than for the mutant construct and likely points to a higher loading capacity for each vesicle. From these observations it is apparent that the total content of the vesicle is not deposited into the PM during this brief interaction. Differences in the fraction of receptors deposited per interaction could contribute to the reduced agonist-induced currents.

Persistent events were also observed. Some insertions lasted for more than a second and in the final frames can be seen to grow dimmer as the receptors are diluted by diffusion away from the insertion point (FIG. 4 of Richards et al.). During these events, vesicles evidently deposit their entire cargo of receptors into the PM. We observed no differences in the frequency of vesicles exhibiting sustained interaction when we compared the mutant and wt receptors. The presence of ERES in the processes of neurons indicates that receptors are likely assembled in the dendritic ER and subsequently inserted. The contrast between the duration of insertion events suggests that the more transient, but more frequent, interaction likely deposits only a small portion of the receptors contained in the vesicle.

Nicotine Incubations Increased PM α4β2 but not α4β4 nAChR Insertions

α4β4* nAChRs are expressed at relatively low levels in the brain, for instance in the medial habenula-interpeduncular nucleus pathway (44). As such, little work has been done on the upregulation of these receptors as compared to α4β2. ALS has been linked to the degeneration of neurons in the cortex as well as motor neurons.

In order to determine if chronic nicotine has a differential affect on α4β2 and α4β4 receptors, we exposed cortical neurons expressing each set of subunits to 200 nM nicotine for 24 hr. Under these conditions, nicotine does not affect the number of insertions events for α4β4. Several previous studies show that α4β4 nAChRs are not substantially upregulated at the PM by nicotine. A constant population of receptors at the PM could potentially mask similar rates of insertion and endocytosis. Our data show, however, that the simplest explanation is probably correct: chronic incubation in nicotine does not alter the insertion of α4β4 nAChRs.

On the other hand, α4β2 nAChRs exhibited an almost 2-fold increase in the number of insertion events in the presence of chronic nicotine. Recently nicotine has been shown to result in an increase in ERES in Neuro-2a cells transfected with α4β2. Interestingly, we have also now shown that the β4R348C mutation results in a reduction of ERES which correlates with a reduction in the trafficking of receptors to the PM. Nicotine clearly affects the trafficking of α4β2 supporting the hypothesis that it acts as a pharmacological chaperone to increase the number of receptors in the ER, which correspondingly leads to increased ERES. The increased trafficking from the ER results in higher expression at the PM, which has been widely shown in both fluorescence and electrophysiological based studies. Based solely on our trafficking studies, our data do not support the idea that α4β4 undergoes upregulation or that nicotine acts as a chaperone for these receptors.

In summary our results indicate that the point mutation in the M3-M4 loop of β4 alters the trafficking of these receptors early in the secretory pathway. On the other hand, α4β2, but not α4β4 insertions are affected by nicotine. The results provide insight into the trafficking of nAChRs and suggest a potential mechanism for the role of nicotinic receptors in ALS.

### REFERENCES

The references cited in Example 4 are listed in Richards, et al., 2011, J. Biol. Chem. 286(37):31241-31249, the entire contents of which are incorporated herein by reference.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of ameliorating an endoplasmic reticulum (ER) stress response in cells expressing nicotinic acetylcholine receptors (nAChRs), the method comprising contacting cells expressing nAChRs with an effective amount of a ligand for nAChRs.

2. The method of claim 1, wherein the contacting results in attenuating endogenously expressed ATF6 translocation, expression of XBP1, phosphorylation of eukaryotic initiation factor 2α (peIF2α), increased numbers of ER exit sites, and/or inhibits upregulation of CCAAT/Enhancer-Binding Protein Homologous Protein (CHOP) levels in the cells.

3. The method of claim 1, wherein the cells are central nervous system (CNS) neurons.

4. The method of claim 3, wherein the neurons are thalamic, cortical, habenular, substantia nigra (SN) or motor neurons.

5. The method of claim 1, wherein the cells comprise a clonal mammalian cell line selected from the group consisting of HEK or a clone derived therefrom, Neuro2a or a clone derived therefrom, SH-Sy5Y or a parent clone or a clone derived therefrom.

6. The method of claim 1, wherein the cells are derived from embryonic cells or from induced pluripotent stems cells.

7. The method of claim 1, wherein the ligand is an α4β2* agonist, partial agonist, or antagonist.

8. The method of claim 1, wherein the ligand is a positive or negative α4β2* allosteric modulator.

9. The method of claim 1, wherein the ligand binds to intracellular and/or plasma membrane nAChRs.

10. The method of claim 1, wherein the nAChR is an α6β2* nAChR.

11. The method of claim 7, wherein the agonist is selected from the group consisting of cytisine, varenicline, galanthamine, CP-601927, ABT-089, A-85380, (±)-epibatidine, (−)-nicotine, lobeline, sazetidine-A, and derivatives thereof.

12. The method of claim 1, wherein the cells are in or derived from a patient having, or at risk of developing, a neurodegenerative disorder.

13. The method of claim 9, wherein the disorder is amyotrophic lateral sclerosis (ALS), Parkinson's disease, Alzheimer's disease, or cognitive deficiency.

14. The method of claim 13, wherein the contacting comprises transdermal administration, oral administration, intrathecal administration, intramuscular administration, intraperitoneal administration, intranasal administration, intravenous administration, suppository, inhalation, or subcutaneous administration.

15. The method of claim 1, wherein the effective amount of ligand is less than 10% of the amount required to activate the nAChRs.

16. The method of claim 1, wherein the effective amount of ligand is less than 1% of the amount required to activate the nAChRs.

17. The method of claim 1, wherein the effective amount of ligand is less than 0.1% of the amount required to activate the nAChRs.

18. The method of claim 1, wherein the contacting occurs for at least two weeks.

19. The method of claim 1, wherein the contacting comprises intracellular binding of the ligand to nAChRs in endoplasmic reticulum or cis-Golgi apparatus.

20. A method of screening for neural pharmacotherapeutic agents, the method comprising:
(a) contacting a candidate agent with a cell modified to express an α4β2 nAChR; and
(b) measuring an indicator of ER stress, the indicator selected from expression and/or translocation in the cell of AFT6, phosphorylation of eIF2α, expression of XBP1, and/or increased numbers of ER exit sites
wherein a candidate agent that evokes an increase in an indicator of ER stress is identified as a neural pharmacotherapeutic agent.

21. A method of treating a neurodegenerative disorder in a subject, the method comprising administering to the subject a ligand an α4β2* agonist, partial agonist, antagonist or allosteric modulator.

22. The method of claim 21, wherein the neurodegenerative disorder is ALS.
